(19) United States
(12) Patent Application Publication
(10) Pub. No.: US 2009/0170872 A1
(43) Pub. Date: Jul. 2, 2009

(54) Compounds and Their Pharmaceutical Use

(75) Inventors: Ganapavarapu Veera Raghava Sharma, Tamilnadu (IN); Narayanan Sukunath, Tamilnadu (IN); Thirunavukkarasu Saravanan, Tamilnadu (IN); Pichika Nagalakshmi, Tamilnadu (IN); Rajagopal Sriram, Tamilnadu (IN); Mani Kamaraj, Tamilnadu (IN)

Correspondence Address: BIRCH STEWART KOLASCH & BIRCH, PO BOX 747, FALLS CHURCH, VA, 22040-0747, US

(73) Assignee: Orchid Research Laboratories Limited, Chennai (IN)

(21) Appl. No.: 11/988,303

(22) PCT Filed: Jul. 4, 2006
(86) PCT No.: PCT/IB2006/001843

§ 371 Date: May 15, 2008

(30) Foreign Application Priority Data
Jul. 5, 2005 (IN) 873/CHE/2005

(51) Int. Cl. A61K 31/497; C07D 403/02; A61K 31/415; C07D 401/14; A61K 31/4439; C07D 403/14

(52) U.S. Cl. 514/255.05; 548/311.4; 514/397; 546/276.7; 514/339; 544/405

(57) ABSTRACT

The present invention relates to novel compounds of the general formula (I), their analogs, their stereoisomers, their polymorphs, their hydrates, their solvates, their pharmaceutically acceptable salts and compositions. The present invention more particularly provides novel compounds of the general formula (I).

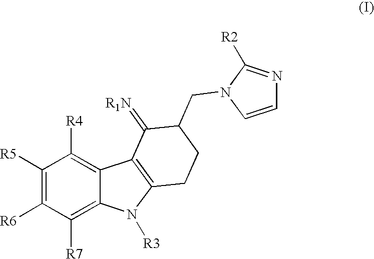

The following specification particularly describes the nature of the invention and the manner in which it has to be performed;

### FIELD OF INVENTION

The present invention relates to novel compounds of the general formula (I), their analogs, their stereoisomers, their polymorphs, their hydrates, their solvates, their pharmaceutically acceptable salts and compositions. The present invention more particularly provides novel compounds of the general formula (I).

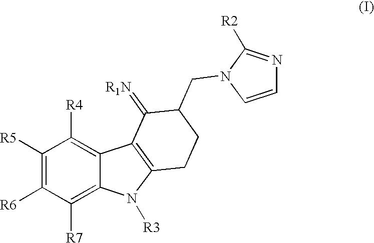

The present invention also provides a process for the preparation of the above said novel compounds of the formula (I), their derivatives, their analogs, their stereoisomers, their polymorphs, their hydrates, their solvates, their pharmaceutically acceptable salts, and compositions.

### BACKGROUND OF INVENTION

The novel compounds of the present invention are useful for a new treatment of inflammations of the respiratory tract. PCT/EP00/07487 discloses a new use for compounds having 5-HT3 (Serotonin M) receptor activity, in particular 5-HT3-receptor specific antagonist activity, for a new treatment of inflammations of the respiratory tract. It also discloses that 5-HT3 receptor antagonists are useful for the treatment of inflammatory diseases of the respiratory tract, especially obstructive pulmonary/bronchial diseases, or laryngospasm. Also the novel compounds of the present invention are useful for a new treatment of various TNF-α mediated diseases as described below. Cytokines are molecules secreted by the immune cells that are important in mediating immune responses. Cytokine production may lead to the secretion of other cytokines, altered cellular function, cell division or differentiation. Inflammation is the body's normal response to injury or infection. The cytokine tumor necrosis factor-alpha (TNF-α) plays a central role in the inflammatory response and has been targeted as a point of intervention in inflammatory disease. TNF-α participates in the protective inflammatory response by activating leukocytes and promoting their migration to extra vascular sites of inflammation (Moser et al., J Clin Invest, 83, 444-55, 1989). At higher concentrations, TNF-α can act as a potent pyrogen and induce the production of other pro inflammatory cytokines (Haworth et al., Eur J Immunol., 21, 2575-79, 1991; Brennen et al., Lancet, 2, 244-7, 1989). TNF-α also stimulates the synthesis of acute-phase proteins. In rheumatoid arthritis, a chronic and progressive inflammatory disease affecting about 1% of the adult U.S. population, TNF-α mediates the cytokine cascade that leads to joint damage and destruction (Arend et al., Arthritis Rheum, 38, 151-60, 1995). Inhibitors of TNF-α, including soluble TNF receptors (etanercept) (Goldenberg, Clin Ther, 21, 75-87, 1999) and anti-TNF-α antibody (infliximab) (Luong et al., Annn Pharmacother, 34, 743-60, 2000), have recently been approved by the U.S. FDA as agents for the treatment of rheumatoid arthritis.

Elevated levels of TNF-α and/or IL-1, over the basal levels have been implicated in mediating or exacerbating a number of disease states including asthma, rheumatoid arthritis, osteoporosis; multiple myeloma; uveititis; acute and chronic myelogenous leukemia; pancreatic-β-cell destruction; osteoarthritis; rheumatoid spondylitis; gouty arthritis; inflammatory bowel disease; allergic rhinitis; ulcerative colitis; anaphylaxis; contact dermatitis; muscle degeneration; cachexia; type I and type II diabetes; bone resorption diseases; ischemia reperfusion injury; atherosclerosis; brain trauma; multiple sclerosis; cerebral malaria; sepsis; septic shock; toxic shock syndrome; fever, and myalgias due to infection. HIV-1, HIV-2, HIV-3, cytomegalovirus (CMV), influenza, adenovirus, the herpes viruses (including HSV-1, HSV-2), and herpes zoster are also exacerbated by TNF-α. Elevated levels of TNF-α have also been implicated in many other disorders and disease conditions, including cachexia, septic shock syndrome, osteoarthritis, inflammatory bowel disease such as Crohn's disease and ulcerative colitis etc. It can be seen that inhibitors of TNF-α are potentially useful in the treatment of a wide variety of diseases. Compounds that inhibit TNF-α have been described in several patents.

Excessive production of IL-6 is implicated in several disease states; it is highly desirable to develop compounds that inhibit IL-6 secretion. Compounds that inhibit IL-6 have been described in U.S. Pat. Nos. 6,004,813; 5,527,546 and 5,166,137.

The cytokine IL-1β also participates in the inflammatory response. It stimulates thymocyte proliferation, fibroblast growth factor activity, and the release of prostaglandin from synovial cells. Elevated or unregulated levels of the cytokine IL-1β have been associated with a number of inflammatory diseases and other disease states, including but not limited to adult respiratory distress syndrome, allergy, Alzheimer's disease etc. Since overproduction of IL-1β is associated with numerous disease conditions, it is desirable to develop compounds that inhibit the production or activity of IL-1β.

In rheumatoid arthritis models in animals, multiple intra-articular injections of IL-1 have led to an acute and destructive form of arthritis (Chandrasekhar et al., Clinical Immuno Immunopathol. 55, 382, 1990). In studies using cultured rheumatoid synovial cells, IL-1 is a more potent inducer of stromelysin than TNF-α. (Firestein, Am. J. Pathol. 140, 1309, 1992). At the sites of local injection, neutrophil, lymphocyte, and monocyte emigration has been observed. The emigration is attributed to the induction of chemokines (e.g., IL-8), and the up-regulation of adhesion molecules (Dinarello, Eur. Cytokine Netw. 5,517-531, 1994).

In rheumatoid arthritis, both IL-1 and TNF-α induce synoviocytes and chrondrocytes to produce collagenase and neutral proteases, which leads to tissue destruction within the arthritic joints. In a model of arthritis (Collagen-induced arthritis (CIA) in rats and mice) intra-articular administration of TNF-α either prior to or after the induction of CIA led to an accelerated onset of arthritis and a more severe course of the disease (Brahn et al., Lymphokine Cytokine Res. 11, 253, 1992; and Cooper, Clin. Exp. Immunol. 898, 244, 1992).

IL-8 has been implicated in exacerbating and/or causing many disease states in which massive neutrophil infiltration into the sites of inflammation or injury (e.g., ischemia) is mediated. Chemotactic nature of IL-8, is included, but not limited to, the following: asthma, inflammatory bowel disease, psoriasis, adult respiratory distress syndrome, cardiac and renal reperfusion injury, thrombosis and glomerulonephritis. In addition to the chemotaxis effect on neutrophils, IL-8 also has the ability to activate neutrophils. Thus, reduction in the IL-8 levels may lead to diminished neutrophil infiltration.

WO 00/64441 discloses the invention which relates to a compound having agonist activity to the 5-HT3 receptor for use as a medicarnent, in therapeutic or prophylactic treatment of disorders involving bronchocontraction of a human or animal body, as well as methods of treatment, wherein said compounds are administered. The invention disclosed in the same patent also relates to a compound having antagonist activity to the 5-HT2a receptor for use as a medicament in therapeutic or prophylactic treatment of disorders involving bronchocontraction of a human or animal body, as well as methods of treatment, wherein the said compounds are administered.

Inhaled 5-hydroxytryptamine (5-HT) causes bronco constriction in asthmatics, and 5-HT plasma levels are elevated in asthma. Electrical field stimulation (EFS) of human airways, in vitro, evokes cholinergic contraction mediated by the release of acetylcholine (Ach) from postganglionic nerves (Eur Respir. J., 1999, 14, 642-649). The same publication also describes about the investigation of whether selective 5-HT agonists and antagonists can modulate EFS-induced cholinergic contraction in human airways in vitro. Increased levels of free 5-HT have been shown to be present in the plasma of symptomatic asthmatic patients compared with the levels in asymptomatic patients (TiPS: Trends in Pharmacological Sciences, January 2000, vol 21, p. 13). In addition, free 5-HT has been shown to correlate positively with the clinical status and negatively with the pulmonary function. These findings suggest that 5-HT might play a role in the pathophysiology of acute asthma. Accordingly, modifiers of the 5-HT transmitter system such as compounds that affect the 5-HT transporter, prejunctional 5-HT receptors or postsynaptic 5-HT receptors might represent a novel treatment of asthma.

Few Prior Art References, which Disclose the Closest Compounds, are Given Here:

I. U.S. Pat. No. 4,695,578 and EP 0221629B1 disclose the structure:

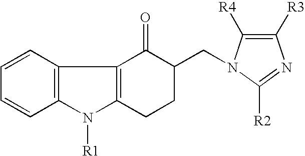

Wherein, R₁ represents a H atom, C₁₋₁₀ alkyl, C₃₋₇ cycloalkyl, C₃₋₆ alkenyl, C₃₋₇ cycloalkyl-C₁₋₄ alkyl, C₃₋₁₀ alkynyl, phenyl, phenyl-C₁₋₃ alkyl group, and one of the groups R₂, R₃ and R₄ is a hydrogen atom or C₁₋₆ alkyl, C₃₋₇ cycloalkyl, C₂₋₆ alkenyl or, phenyl-C₁₋₃ alkyl group and each of the other two groups which may be same or different represents a H atom, C₁₋₆ alkyl group and physiologically acceptable salts and solvates, example hydrates and thereof.
II. GB 2202530A discloses the structure:

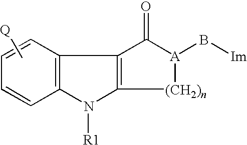

Wherein, Im represents the imidazolyl group of the formula

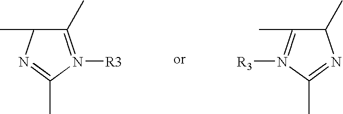

R₁ represents a H atom, C₁₋₆ alkyl, C₃₋₇ cycloalkyl, C₃₋₆ alkenyl, C₃₋₇ cycloalkyl-C₁₋₄ alkyl, C₃₋₁₀ alkynyl, phenyl, phenyl-C₁₋₃ alkyl group, —CO₂R₅, —COR₅, —CONR₅NR₆ or —SO₂R₅ (wherein R₅ and R₆ may be same or different, and each represents a H atom, C₁₋₆ alkyl, C₃₋₇ cycloalkyl or a phenyl, phenyl-C₁₋₄ alkyl group wherein the phenyl group is optionally substituted by one or more C₁₋₄ alkyl, C₁₋₄ alkoxy or hydroxy groups or halogen atoms, with the proviso that R₅ doesn't represent a H atom when R₁ represents a group —CO₂R₅ or —SO₂R₅) and one of the groups R₂, R₃ and R₄ is a hydrogen atom or C₁₋₆ alkyl, C₃₋₇ cycloalkyl, C₃₋₄ alkenyl, phenyl or phenyl-C₁₋₃ alkyl group and each of the other two groups which may be same or different represents a H atom, C₁₋₆ alkyl group; Q represents a H atom or a halogen atom, or a hydroxy, C₁₋₄ alkyl or C₃₋₄ alkenyl group or together with the N atom to which they are attached, form a saturated 5 to 7 membered ring; n represents 1, 2 or 3; and A-B represents the group —CH—CH₂ or —C═CH; and physiologically acceptable salts and solvates thereof.

### OBJECTIVE OF THE INVENTION

The objective of the present invention is to disclose novel compounds showing TNF-□ and IL-6 inhibition. TNF-□ is a proinflammatory cytokine and plays a role in inflammatory and immunological events. The major sources of TNF-□ are mast cells, eosinophils, macrophages, and monocytes. TNF-□ causes a broad spectrum of effects both in vitro and in vivo, including vascular thrombosis and tumor necrosis, inflammation, activation of macrophages and neutrophils, leukocytosis, apoptosis, and shock. TNF-□ has been associated with a variety of disease states including various forms of cancer, arthritis, psoriasis, endotoxic shock, sepsis, autoimmune diseases, infarctions, obesity, asthma, COPD, cachexia, stroke, glaucoma, retinitis, atherosclerosis and uveitis. Also the objective of the present invention is to disclose the compounds likely to act as competitive antagonists of serotonin receptor subtype 5-HT3 present in vitro and in vivo in the gastrointestinal, brain, and other tissues, and also as potent anti emetic agents.

### SUMMARY OF THE INVENTION

The present invention relates to novel compounds of the formula (I),

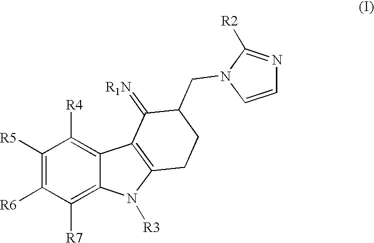

their derivatives, their analogs, their stereoisomers, their polymorphs, their hydrates, their solvates, their pharmaceutically acceptable salts, and compositions, wherein R₁ represents —O(CH₂)ₙR₈, where R₈ represents hydrogen, substituted or unsubstituted groups selected from alkyl, alkenyl, alkynyl, aryl, cycloalkyl, heterocyclyl, heteroaryl, haloalkyl, or a counter ion, —C(═O)R₉, where R₉ represents hydrogen, hydroxyl, substituted or unsubstituted groups selected from alkyl, alkenyl, alkynyl, alkoxy, amino, aryl, aryloxy, arylalkoxy, arylalkyl, arylalkynyl, haloalkyl, heteroaryl, heteroaryloxy, heteroarylalkoxy, heteroarylalkyl, (heteroaryl)alkenyl, heterocyclyl, (heterocyclyl)alkenyl, (heterocyclyl)alkynyl, cycloalkyl, cycloalkyloxy; R₂ represents hydrogen, hydroxyl, alkyl, haloalkyl, halogen, mono or di alkylamino, nitro, alkoxy, thiol, alkylthio, aryl, aralkyl, arylthio, heteroaryl, heteroaralkyl, and cycloalkyl; R₃ represents hydrogen, hydroxyl, nitro, nitroso, halogen, optionally substituted groups selected from alkyl, haloalkyl, mono or dialkylamino, alkoxy, arylalkyl, aryl, aryloxy; R₄, R₅, R₆ and R₇ may be same or different and independently represents hydrogen, nitro, hydroxyl, formyl, azido, cyano, halo, or optionally substituted groups selected from alkyl, aryl, heteroaryl, alkoxy, haloalkyl, hydrazino, monoalkylamino, dialkylamino, alkylsulfonyl, alkylsulfinyl, arylsulfonyl, arylsulfinyl, alkylthio, arylthio, arylalkyl, alkoxyalkyl, sulfamoyl, carboxylic acid and its derivatives; n is an integer ranging from 0 to 2.

### DETAILED DESCRIPTION OF THE INVENTION

Suitable groups represented by R¹ represents —O(CH₂)ₙR₈ where R₈ represents hydrogen; substituted or unsubstituted groups selected from (C₁-C₄) alkyl groups, such as methyl, ethyl, n-propyl, isopropyl, t-butyl and the like; alkenyl groups such as ethylene and the like, the alkenyl group may be substituted; alkynyl groups such as acetylene and the like, the alkynyl group may be substituted; aryl groups such as phenyl, naphthyl and the like, the aryl group may be substituted; aralkyl groups such as benzyl, phenylethyl, phenylpropyl and the like; the aralkyl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like; the heteroaryl group may be substituted; haloalkyl groups selected from chloromethyl, chloroethyl, trifluoromethyl, trifluoroethyl, dichloromethyl, dichloroethyl and the like, the haloalkyl group may be substituted; cycloalkyl groups such as cyclopropyl, cyclobutyl, cyclopentyl and the like, the cycloalkyl group may be substituted; heterocyclyl containing at least one heteroatom selected from the O, N, S such as piperidine, piperazine, morpholine, 1,4-dioxane and the like, the heterocyclyl group may be substituted, or a counter ion, when R₈ represents —C(═O)R₉, therein R₉ represents hydrogen, hydroxyl, substituted or unsubstituted groups selected from (C₁-C₄) alkyl groups such as methyl, ethyl, n-propyl, isopropyl and the like; alkenyl groups such as ethylene and the like, the alkenyl group may be substituted; alkynyl groups such as acetylene and the like, the alkynyl group may be substituted; linear or branched (C₁-C₆) alkoxy groups, such as methoxy, ethoxy, n-propoxy, isopropoxy and the like, amino groups such as methyl amine, ethyl amine, isopropylamine, (N,N)-dimethyl amine and the like, aryl groups such as phenyl, naphthyl and the like, the aryl group may be substituted; arylalkoxy groups such as phenylmethoxy, phenylethoxy, phenylpropoxy, and the like; arylalkyl groups such as benzyl, phenylethyl, phenylpropyl and the like; aryl(C₂-C₆) alkenyl, aryl(C₂-C₆) alkynyl, (C₃-C₇) cycloalkyl, haloalkyl groups selected from chloromethyl, chloroethyl, trifluoromethyl, trifluoroethyl, dichloromethyl, dichloroethyl and the like; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like, heteroarylalkoxy, heteroarylalkyl, heteroarylalkenyl wherein the alkenyl group is selected from ethylene and the like, and the hetero aryl part is selected from pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like, the heteroaryl group may be substituted; heteroaryl alkynyl, hetereoaryloxy, heterocyclyl, (heterocyclyl)alkenyl, (heterocyclyl)alkynyl wherein the heterocycle contains at least one hetroatom selected from the O, N, S such as piperidine, piperazine, pyrazine, morpholine, 1,4-dioxane and the like, (C₃-C₇) cycloalkyl groups such as cyclopropyl, cyclobutyl, cyclopentyl and the like.

R₂ represents hydrogen, hydroxyl, alkyl (selected from substituted or unsubstituted (C₁-C₄) alkyl groups, such as methyl, ethyl, n-propyl, isopropyl, t-butyl and the like), haloalkyl, halogen, mono or di alkylamino, nitro, alkoxy, thiol, alkylthio, aryl, aralkyl, arylthio, heteroaryl, heteroaralkyl, and cycloalkyl;

R₃ represents hydrogen, hydroxyl, nitro, nitroso, halogen, optionally substituted groups selected from alkyl (which may be selected from substituted or unsubstituted (C₁-C₄) alkyl groups, such as methyl, ethyl, n-propyl, isopropyl, t-butyl and the like), haloalkyl, mono or dialkylamino, alkoxy, arylalkyl, aryl, aryloxy heteroaryl, heteroaralkyl, cycloalkyl;

R₄, R₅, R₆ and R₇ may be same or different and independently represent hydrogen, nitro, hydroxy, formyl, azido, cyano, halo, or optionally substituted groups selected from alkyl, aryl, alkoxy, haloalkyl, hydrazine, monoalkylamino, dialkylamino, alkylsulfonyl, alkylsulfinyl, arylsulfonyl, arylsulfinyl, alkylthio, arylthio, arylalkyl, alkoxyalkyl, sulfamoyl, carboxylic acid and its derivatives;

n is an integer ranging from 0 to 2.

When the aryl and heteroaryl groups representing R₈ and R₉ are substituted by one or more substituents which may be same or different, the substituents may be selected from halogens (fluorine, chlorine, bromine, iodine), hydroxy, nitro, cyano, azido, nitroso, amino, hydrazine, formyl, alkyl, haloalkyl, haloalkoxy, cycloalkyl, aryl (may be further substituted), alkoxy, aryloxy, acyl, acyloxy, acyloxyacyl, methylene dioxy, heterocyclyl, heteroaryl (may be further substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives and these substituents are as defined above.

Furthermore, whenever the groups R₈ and R₉ represent substituted or unsubstituted 5 to 10 membered ring systems, the rings may be monocyclic or bicyclic, saturated or partially saturated or aromatic containing 1 to 4 heteroatoms selected from O, S and N.

Pharmaceutically acceptable salts of the present invention include alkali metal salts like Li, Na, and K salts, alkaline earth metal salts like Ca and Mg salts, salts of organic bases such as diethanolamine, α-phenylethylamine, benzylamine, piperidine, morpholine, pyridine, hydroxyethylpyrrolidine, hydroxyethylpiperidine, guanidine, choline and the like, ammonium or substituted ammonium salts, aluminum salts. Salts also include amino acid salts such as glycine, alanine, cysteine, lysine, arginine, phenylalanine etc. Salts may include sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

Particularly Useful Compounds According to the Invention Include:
1. (4E)-9-methyl-3-[(2-methyl-1H-imidazol-1-yl)methyl]-1,2,3,9-tetrahydro-4H-carbazol-4-one oxime;
2. (4E)-9-methyl-3-[(2-methyl-1H-imidazol-1-yl)methyl]-1,2,3,9-tetrahydro-4H-carbazol-4-one O-benzyloxime;
3. (4E)-9-methyl-3-[(2-methyl-1H-imidazol-1-yl)methyl]-1,2,3,9-tetrahydro-4H-carbazol-4-one O-ethyloxime;
4. (4E)-9-methyl-3-[(2-methyl-1H-imidazol-1-yl)methyl]-1,2,3,9-tetrahydro-4H-carbazol-4-one O-2,2,2-trifluoroethyloxime;
5. (4E)-9-methyl-3-[(2-methyl-1H-imidazol-1-yl)methyl]-1,2,3,9-tetrahydro-4H-carbazol-4-one O-(4-methoxybenzyl)oxime;
6. (4E)-9-methyl-3-[(2-methyl-1H-imidazol-1-yl)methyl]-1,2,3,9-tetrahydro-4H-carbazol-4-one O-(allyl)oxime;
7. (4E)-9-methyl-3-[(2-methyl-1H-imidazol-1-yl)methyl]-1,2,3,9-tetrahydro-4H-carbazol-4-one O-(prop-2-ynyl)oxime;
8. (4E)-9-methyl-3-[(2-methyl-1H-imidazol-1-yl)methyl]-1,2,3,9-tetrahydro-4H-carbazol-4-one O-(3-cyano-pyridin-2-yl)oxime;
9. (4E)-9-methyl-3-[(2-methyl-1H-imidazol-1-yl)methyl]-1,2,3,9-tetrahydro-4H-carbazol-4-one O-(4-fluorobenzyl)oxime;
10. (4E)-9-methyl-3-[(2-methyl-1H-imidazol-1-yl)methyl]-1,2,3,9-tetrahydro-4H-carbazol-4-one O-(pyidin-3-ylmethyl)oxime;
11. (4E)-9-methyl-3-[(2-methyl-1H-imidazol-1-yl)methyl]-1,2,3,9-tetrahydro-4H-carbazol-4-one O-(pyridin-4-ylmethyl)oxime;
12. (4E)-9-methyl-3-[(2-methyl-1H-imidazol-1-yl)methyl]-1,2,3,9-tetrahydro-4H-carbazol-4-one O-(3,5-dimethyl-4-methoxy-pyridin-2-ylmethyl)oxime;
13. (4E)-9-methyl-3-[(2-methyl-1H-imidazol-1-yl)methyl]-1,2,3,9-tetrahydro-4H-carbazol-4-one O-(pyridin-2-ylmethyl)oxime;
14. (4E)-9-methyl-3-[(2-methyl-1H-imidazol-1-yl)methyl]-1,2,3,9-tetrahydro-4H-carbazol-4-one O-(4-cyanobenzyl)oxime;
15. (4E)-9-methyl-3-[(2-methyl-1H-imidazol-1-yl)methyl]-1,2,3,9-tetrahydro-4H-carbazol-4-one O-(4-nitrobenzyl)oxime;
16. (4E)-9-methyl-3-[(2-methyl-1H-imidazol-1-yl)methyl]-1,2,3,9-tetrahydro-4H-carbazol-4-one O-(5-nitropyridin-2-yl)oxime;
17. (4E)-9-methyl-3-[(2-methyl-1H-imidazol-1-yl)methyl]-1,2,3,9-tetrahydro-4H-carbazol-4-one O-[5-bromo-pyridin-2-yl]oxime;
18. (4E)-9-methyl-3-[(2-methyl-1H-imidazol-1-yl)methyl]-1,2,3,9-tetrahydro-4H-carbazol-4-one O-[3-cyano-5,6-dimethylpyridin-2-yl]oxime;
19. (4E)-9-methyl-3-[(2-methyl-1H-imidazol-1-yl)methyl]-1,2,3,9-tetrahydro-4H-carbazol-4-one O-[3-nitro-pyridin-2yl]oxime;
20. (4E)-9-methyl-3-[(2-methyl-1H-imidazol-1-yl)methyl]-1,2,3,9-tetrahydro-4H-carbazol-4-one O-(2,6-dimethoxypyrimidin-4-yl)oxime;
21. (4E)-9-methyl-3-[(2-methyl-1H-imidazol-1-yl)methyl]-1,2,3,9-tetrahydro-4H-carbazol-4-one O-[5-trifluoromethyl-pyridin-2-yl]oxime;
22. (4E)-9-methyl-3-[(2-methyl-1H-imidazol-1-yl)methyl]-1,2,3,9-tetrahydro-4H-carbazol-4-one O-(-chloro-5-trifluoromethyl-pyridin-2-yl)oxime.
23. (4E)-9-methyl-3-[(2-methyl-1H-imidazol-1-yl)methyl]-1,2,3,9-tetrahydro-4H-carbazol-4-one O-(3-cyano-6-methylpyridin-2-yl)oxime;
24. (4E)-9-methyl-3-[(2-methyl-1H-imidazol-1-yl)methyl]-1,2,3,9-tetrahydro-4H-carbazol-4-one O-(6-trifluoromethylpyrimidin-2-yl)oxime;
25. (4E)-9-methyl-3-[(2-methyl-1H-imidazol-1-yl)methyl]-1,2,3,9-tetrahydro-4H-carbazol-4-one O-acetyloxime;
26. (4E)-9-methyl-3-[(2-methyl-1H-imidazol-1-yl)methyl]-1,2,3,9-tetrahydro-4H-carbazol-4-one O-trifluoroacetyloxime;
27. (4E)-9-methyl-3-[(2-methyl-1H-imidazol-1-yl)methyl]-1,2,3,9-tetrahydro-4H-carbazol-4-one O-2-chloropyridinoyloxime;
28. (4E)-9-methyl-3-[(2-methyl-1H-imidazol-1-yl)methyl]-1,2,3,9-tetrahydro-4H-carbazol-4-one O-cyclobutanoyloxime;
29. (4E)-9-methyl-3-[(2-methyl-1H-imidazol-1-yl)methyl]-1,2,3,9-tetrahydro-4H-carbazol-4-one O-2-cyclopentanoyloxime;
30. (4E)-9-methyl-3-[(2-methyl-1H-imidazol-1-yl)methyl]-1,2,3,9-tetrahydro-4H-carbazol-4-one O-benzoyloxime;
31. (4E)-9-methyl-3-[(2-methyl-1H-imidazol-1-yl)methyl]-1,2,3,9-tetrahydro-4H-carbazol-4-one O-4-chloro-benzoyloxime;
32. (4E)-9-methyl-3-[(2-methyl-1H-imidazol-1-yl)methyl]-1,2,3,9-tetrahydro-4H-carbazol-4-one O-cyclopropanoyloxime;
33. (4E)-9-methyl-3-[(2-methyl-1H-imidazol-1-yl)methyl]-1,2,3,9-tetrahydro-4H-carbazol-4-one O-2-fluorobenzoyloxime;
34. (4E)-9-methyl-3-[(2-methyl-1H-imidazol-1-yl)methyl]-1,2,3,9-tetrahydro-4H-carbazol-4-one O-4-trifluoromethylphenyloxime;
35. (4E)-9-methyl-3-[(2-methyl-1H-imidazol-1-yl)methyl]-1,2,3,9-tetrahydro-4H-carbazol-4-one O-4-methoxybenzoyloxime;
36. (4E)-9-methyl-3-[(2-methyl-1H-imidazol-1-yl)methyl]-1,2,3,9-tetrahydro-4H-carbazol-4-one O-4-methylbenzoyloxime;
37. (4E)-9-methyl-3-[(2-methyl-1H-imidazol-1-yl)methyl]-1,2,3,9-tetrahydro-4H-carbazol-4-one O-2-chlorobenzoyloxime;
38. (4E)-9-methyl-3-[(2-methyl-1H-imidazol-1-yl)methyl]-1,2,3,9-tetrahydro-4H-carbazol-4-one O-4-fluorobenzoyloxime;
39. (4E)-9-methyl-3-[(2-methyl-1H-imidazol-1-yl)methyl]-1,2,3,9-tetrahydro-4H-carbazol-4-one O-2-bromobenzoyloxime;
40. (4E)-9-methyl-3-[(2-methyl-1H-imidazol-1-yl)methyl]-1,2,3,9-tetrahydro-4H-carbazol-4-one O-6-chloropyridinoyloxime;
41. (4E)-9-methyl-3-[(2-methyl-1H-imidazol-1-yl)methyl]-1,2,3,9-tetrahydro-4H-carbazol-4-one O-4-ethylbenzoyloxime;
42. (4E)-9-methyl-3-[(2-methyl-1H-imidazol-1-yl)methyl]-1,2,3,9-tetrahydro-4H-carbazol-4-one O-cyclohexanoyloxime;
43. (4E)-9-methyl-3-[(2-methyl-1H-imidazol-1-yl)methyl]-1,2,3,9-tetrahydro-4H-carbazol-4-one O-(3-thiophenyl)propenoyloxime;
44. (4E)-9-methyl-3-[(2-methyl-1H-imidazol-1-yl)methyl]-1,2,3,9-tetrahydro-4H-carbazol-4-one O-4-cyanobenzoyloxime;
45. (4E)-9-methyl-3-[(2-methyl-1H-imidazol-1-yl)methyl]-1,2,3,9-tetrahydro-4H-carbazol-4-one O-5-bromo-2-furanoyloxime;
46. (4E)-9-methyl-3-[(2-methyl-1H-imidazol-1-yl)methyl]-1,2,3,9-tetrahydro-4H-carbazol-4-one O-3-quinolinoyloxime;
47. (4E)-9-methyl-3-[(2-methyl-1H-imidazol-1-yl)methyl]-1,2,3,9-tetrahydro-4H-carbazol-4-one O-5-nitro-2-furanoyloxime;
48. (4E)-9-methyl-3-[(2-methyl-1H-imidazol-1-yl)methyl]-1,2,3,9-tetrahydro-4H-carbazol-4-one O-3-methyl-2-furanoyloxime;
49. (4E)-9-methyl-3-[(2-methyl-1H-imidazol-1-yl)methyl]-1,2,3,9-tetrahydro-4H-carbazol-4-one O-3-methyl-2-thiophenoyloxime;
50. (4E)-9-methyl-3-[(2-methyl-1H-imidazol-1-yl)methyl]-1,2,3,9-tetrahydro-4H-carbazol-4-one O-5-methyl-3-isoxazoloyloxime;
51. (4E)-9-methyl-3-[(2-methyl-1H-imidazol-1-yl)methyl]-1,2,3,9-tetrahydro-4H-carbazol-4-one O-5-methyl-2-thiophenoyloxime;
52. (4E)-9-methyl-3-[(2-methyl-1H-imidazol-1-yl)methyl]-1,2,3,9-tetrahydro-4H-carbazol-4-one O-3-methyl-5-isoxazoloyloxime;
53. (4E)-9-methyl-3-[(2-methyl-1H-imidazol-1-yl)methyl)]-1,2,3,9-tetrahydro-4H-carbazol-4-one O-3-thiophenoyloxime;
54. (4E)-9-methyl-3-[(2-methyl-1H-imidazol-1-yl)methyl]-1,2,3,9-tetrahydro-4H-carbazol-4-one O-4-methylphenylacetyloxime;
55. (4E)-9-methyl-3-[(2-methyl-1H-imidazol-1-yl)methyl]-1,2,3,9-tetrahydro-4H-carbazol-4-one O-2-thiophenoyloxime;
56. (4E)-9-methyl-3-[(2-methyl-1H-imidazol-1-yl)methyl]-1,2,3,9-tetrahydro-4H-carbazol-4-one O-5-chloro-2-thiophenoyloxime;
57. (4E)-9-methyl-3-[(2-methyl-1H-imidazol-1-yl)methyl]-1,2,3,9-tetrahydro-4H-carbazol-4-one O-[(4,5-dibromo-thien-2-yl)carbonyl]oxime;
58. (4E)-9-methyl-3-[(2-methyl-1H-imidazol-1-yl)methyl]-1,2,3,9-tetrahydro-4H-carbazol-4-one O-[(6-methylpyrazin-2-yl)carbonyl]oxime;
59. (4E)-9-methyl-3-[(2-methyl-1H-imidazol-1-yl)methyl]-1,2,3,9-tetrahydro-4H-carbazol-4-one O-[(pyridin-2-yl)carbonyl]oxime;
60. (4E)-9-methyl-3-[(2-methyl-1H-imidazol-1-yl)methyl]-1,2,3,9-tetrahydro-4H-carbazol-4-one [(furan-2-yl)carbonyl]oxime;
61. (4E)-9-methyl-3-[(2-methyl-1H-imidazol-1-yl)methyl]-1,2,3,9-tetrahydro-4H-carbazol-4-one O-[(2E)-3-(1,3-benzodioxol-5-yl)prop-2-enoyl]oxime;
62. (4E)-9-methyl-3-[(2-methyl-1H-imidazol-1-yl)methyl]-1,2,3,9-tetrahydro-4H-carbazol-4-one O-[(5-bromothien-2-yl)carbonyl]oxime;
63. (4E)-9-methyl-3-[(2-methyl-1H-imidazol-1-yl)methyl]-1,2,3,9-tetrahydro-4H-carbazol-4-one [(furan-3-yl)carbonyl]oxime;
64. 4-(hydroxyamino)-9-methyl-3-[(2-methyl-1H-imidazol-1-yl)methyl]-2,3,4,9-tetrahydro-1H-carbazole; and
65. (4E)-9-methyl-3-[(2-methyl-1H-imidazol-1-yl)methyl]-1,2,3,9-tetrahydro-4H-carbazol-4-one O-3-cyanobenzoyloxime;

Preferred salts for the list of compounds given above are hydrochloride, hydrobromide, sodium, potassium or magnesium.

According to another feature of the present invention, there is provided a process as shown in the following steps, for the preparation of compounds of formula (I), wherein all the other symbols are as defined earlier.
a) The compound of the formula (II) was converted in step-I, to the compound of formula (III) wherein all the other groups are as defined earlier. The compound of the formula (II) is prepared according to the procedure described in the patent GB 2202530A

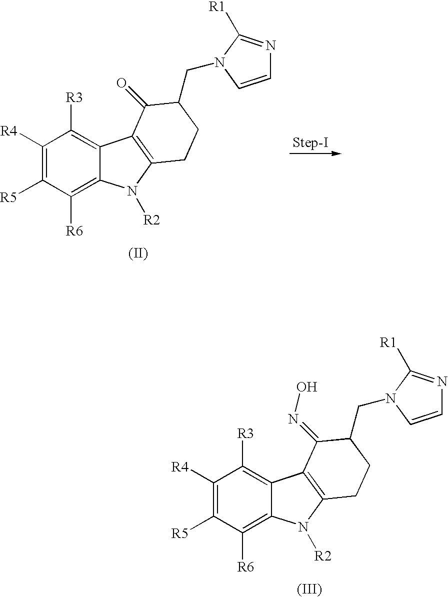

b) Reacted the compound of the formula (III) to give the compound of formula (I) wherein all the other symbols are as defined earlier.

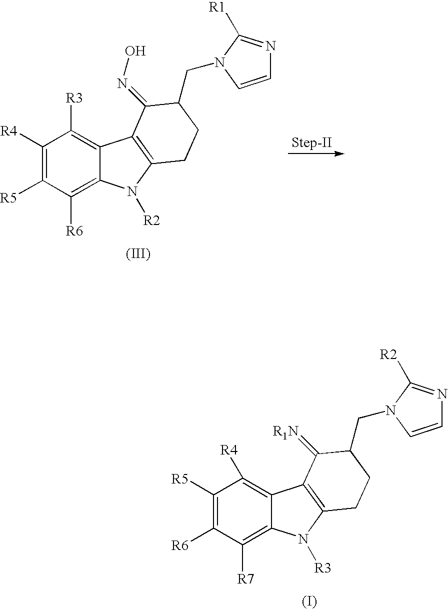

c) Optional reduction of the compound of formula (I) furnished the compound of formula (IV) wherein all the other symbols are as defined earlier.

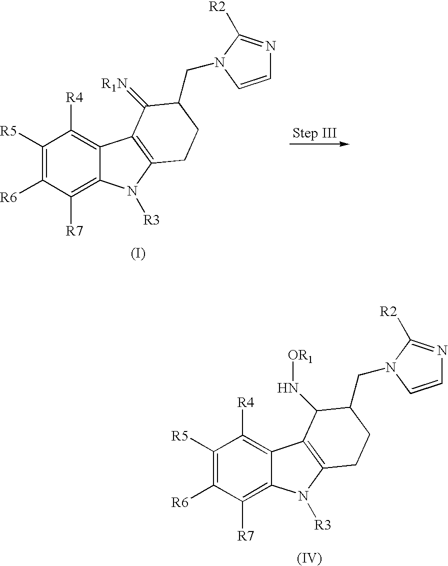

The reactions described in the processes outlined above are performed by using the methods described herein:

Step-I: The compound of formula (II) is converted to its oxime with either hydroxylamine or hydroxylamine hydrochloride in solvents such as methanol, ethanol, isopropanol, n-propanol, n-butanol or a mixture thereof, in the presence of a base like triethylamine, pyridine, DMAP and the like. The reaction is carried out at a temperature in the range of room temperature to reflux temperature (25° C. to 150° C.).
Step-II: The compound of formula (III) is converted to compound of formula (I) in the presence of solvents selected from dichloromethane, chloroform dioxane, dimethylformamide, DMSO, dioxane, diethyl ether, diisopropylether or a mixture thereof, in the presence of a base like sodium hydroxide, sodium hydride, sodium methoxide, sodium ethoxide, sodium t-butoxide and the like.
Step-III: The compound of formula (I) can be optionally converted into the compound of formula (IV) in the presence of solvents selected from THF, diethyl ether, dioxane, and the like, using reducing agents such as borane-pyridine, borane-THF, borane-ether, borane-dioxane, or other reducing agents such as sodium borohydride, lithium aluminum hydride.

The pharmaceutical composition may be in the forms normally employed, such as tablets, capsules, powders, syrups, solutions, aerosols, suspensions and the like, may contain flavoring agents, sweeteners etc. in suitable solid or liquid carriers or diluents, or in suitable sterile media to form injectable solutions or suspensions. Such compositions typically contain from 1 to 20%, preferably 1 to 10% by weight of active compound, the remainder of the composition being pharmaceutically acceptable carriers, diluents or solvents.

The invention is explained in detail in the examples given below which are provided by way of illustration only and therefore should not be construed to limit the scope of the invention.

### Example 1

### Synthesis of (4E)-9-methyl-3-[(2-methyl-1H-imidazol-1-yl)methyl]-1,2,3,9-tetrahydro-4H-carbazol-4-one oxime

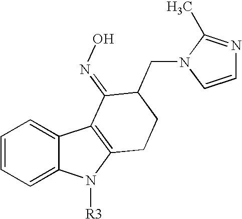

To a slurry of Ondansetron hydrochloride (5 g, 13.6 mmoles) in a mixture of pyridine:methanol (1:2.20 ml,) was added hydroxylamine hydrochloride (5 g, 71 mmoles). The resulting slurry was stirred at 80° C. for 24 hours. Subsequently the reaction mixture was cooled and filtered to yield, a white crystalline solid, which on drying at high vacuum gave the desired compound (3.5 g, 83.5%). R_{f} 0.7 (9:1, dichloromethane:methanol), HPLC (purity): 97%, mp 231-233° C.; ¹H-NMR (CDCl₃) δ (ppm): 8.01 (d, 1H), 7.33-7.25 (m, 3H), 7.21-7.17 (m, 2H), 6.95 (d, 1H), 4.18-4.01 (m, 3H), 3.71 (s, 3H), 2.62 (s, 3H), and 2.25-2.20 (m, 2H); IR (cm⁻¹) 3138.1, 2934.6, 2835.9, 1621.9, 1474.8, and 1278.1; MS m/z: 309.2 [M+1]

### Example 2

### Synthesis of (4E)-9-methyl-3-[(2-methyl-1H-imidazol-1-yl)methyl]-1,2,3,9-tetrahydro-4H-carbazol-4-one O-benzyloxime

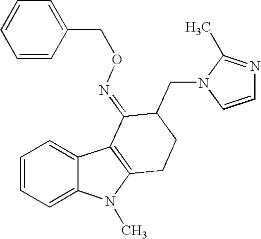

To a solution of O-Benzyl hydroxylamine hydrochloride (0.3 g, 1.87 mmoles) in a mixture of pyridine:methanol (1:1, 10 ml) was added Ondansetran hydrochloride in portions (0.3 g, 0.82 mmoles). The resulting slurry was stirred at 120° C. for 36 hours. After complete conversion, the solvent was evaporated and resulting residue was re-dissolved in 20 ml of chloroform, followed by 20 ml of water. The organic layer was separated, dried over anhydrous sodium sulfate and evaporated at reduced pressure to yield a glassy brown solid, which was then subjected to silica gel column chromatography using a gradient of methanol in ethyl acetate (0-11%), to yield the product (168 mg, 51.9%), R_{f}=0.5 (9:1 chloroform:methanol); HPLC (purity): 94.5%; ¹H-NMR (CDCl₃) δ (ppm): 8.16 (d, 1H), 7.46 (d, 1H), 7.38-7.25 (m, 7H), 6.92 (s, 1H), 6.76 (s, 1H), 5.23 (s, 2H), 4.13-4.08 (m, 2H), 3.92-3.89 (m, 1H), 3.69 (s, 3H), 2.87-2.83 (m, 2H), 2.29 (s, 3H), and 1.98-1.90 (m, 2H); MS m/z: 399.3 [M+1]

### The Following Compounds are Prepared According to the Procedure Given in the Example 2.

| Exp. | Structure | Analytical data |
|---|---|---|
| 3 | 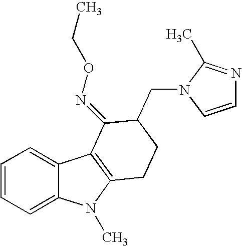 | R_{f} = 0.7 (9:1 CH₂Cl₂: CH₃OH); HPLC (purity): 97%; ¹H-NMR (CDCl₃) δ (ppm): 8.15 (d, 1 H), 7.33-7.21 (m, 3 H), 7.21-7.17 (m, 2 H), 6.92 (d, 1 H), 6.85 (d, 1 H), 4.25 (d, 1 H, 4.24 (d, 1 H), 3.71 (s, 2 H), 3.69 (s, 3 H), 2.87 (m, 2 H), 2.47 (s, 3 H), 2.01 (m, 4 H), and 1.37 (t, 3 H); MS m/z: 337.2 [M + 1] |
| 4 | 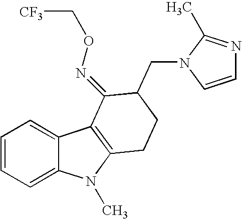 | R_{f} 0.5 (9:1 CHCl₃: CH₃OH); HPLC (purity): 93.7%; ¹H-NMR (CDCl₃) δ (ppm): 8.06 (d, 1 H), 7.31-7.23 (m, 3 H), 6.95 (s, 1 H), 6.85 (s, 1 H), 4.57 (m, 2 H), 4.15 (dd, 2 H), 3.92-3.88 (m, 1 H), 3.72 (s, 3 H), 2.95-2.88 (m, 2 H), 2.47 (s, 3 H), and 2.00-1.96 (m, 2 H); MS m/z: 391.1 [M + 1] |
| 5 | 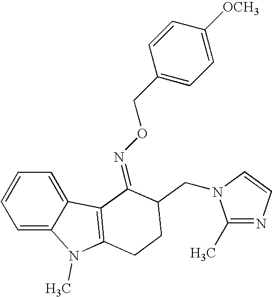 | R_{f} 0.73 (Chloroform: MeOH (9:1); HPLC (purity): 89.6%; ¹H-NMR (CDCl₃) δ (ppm): 1.91-1.94 (m, 2 H), 2.26 (s, 3 H), 2.83-2.86 (m, 2 H), 3.61 (s, 3 H), 3.80 (s, 3 H), 3.86-3.88 (m, 2 H), 4.06-4.11 (m, 1 H), 5.17 (s, 2 H), 6.77 (s, 1 H), 6.87-6.90 (m, 3 H), 7.25-7.28 (m, 3 H), 7.41 (d, 2 H), and 8.18 (t, 1 H); MS m/z: 429.2 (M⁺) |
| 6 | 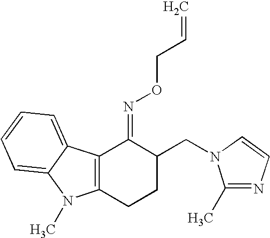 | R_{f} 0.76 (Chloroform: MeOH (9:1), HPLC (purity): 98.2%; m.p - 120-124° C.; ¹H-NMR (CDCl₃) δ (ppm): 1.97-2.03 (m, 2 H), (m, 2 H), 2.49 (s, 3 H), 2.87-2.89 (m, 2 H), 3.70 (s, 3 H), 3.78-3.82 (m, 2 H), 4.15-4.19 (m, 1 H), 4.67-4.69 (d, 2 H), 5.25-5.28 (m, 1 H), 5.36-5.40 (m, 1 H), 6.09-6.11 (m, 1 H), 6.86 (s, 1 H), 6.96 (s, 1 H), 7.22- 7.32 (m, 3 H), and 8.12 (d, 1 H); MS m/z: 349.4 (M⁺) |
| 7 | 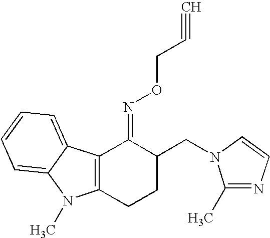 | R_{f} 0.65 (Chloroform: MeOH (9:1); HPLC (purity): 97.3%; ¹H-NMR (CDCl₃) δ (ppm): 1.95-2.04 (m, 2 H), 2.47-2.49 (m, 3 H), 2.86-2.89 (m, 2 H), 3.70 (s, 3 H), 3.77-3.80 (m, 1 H), 3.88-3.92 (m, 1 H), 4.18-4.21 (m, 1 H), 4.80 (d, 2H), 6.86 (d, 1 H), 6.94 (s, 1 H), 7.23-7.30 (m, 4 H), and 8.15 (d, 1 H); MS m/z: 347.2 (M⁺) |
| 8 | 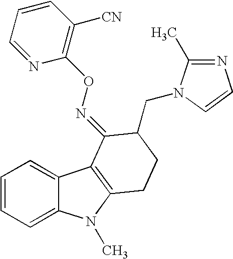 | R_{f} 0.55 (Chloroform: MeOH (9:1); HPLC (purity): 98.8%: ¹H-NMR (CDCl₃) δ (ppm): 1.95-2.04 (m, 2 H), 2.46 (s, 3 H), 2.87-2.92 (m, 2 H), 3.73 (s, 3 H), 3.94-4.00 (m, 1 H), 4.27-4.35 (m, 2 H), 6.88 (d, 1 H), 7.05 (d, 1 H), 7.12-7.15 (m, 1 H), 7.29-7.33 (m, 3 H), 7.97-7.99 (m, 1 H), 8.15 (d, 1 H), and 8.56 (t, 1 H); MS m/z: 411.2 (M⁺) |
| 9 | 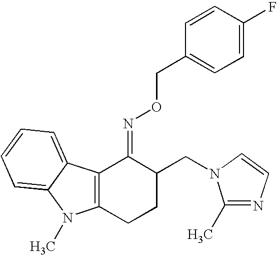 | R_{f} 0.71 (Chloroform: MeOH (9:1); HPLC (purity): 94.5%; ¹H-NMR (CDCl₃) δ (ppm): 1.95-2.04.(m, 2 H), 2.33 (s, 3 H), 2.83-2.87 (m, 2 H), 3.86 (s, 3 H), 3.88-3.92 (m, 2 H), 4.06-4.10 (m, 1 H), 5.18 (s, 2 H), 6.78 (s, 1 H), 7.02 (s, 1 H), 7.04-7.07 (m, 2 H), 7.12-7.15 (m, 1 H), 7.23-7.30 (m, 2 H), 7.43-7.46 (m, 2 H), and 8.12 (d, 1 H); MS m/z: 417.2 (M⁺) |
| 10 | 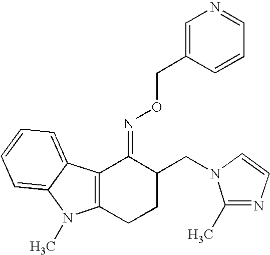 | R_{f} 0.46 (Chloroform: MeOH (9:1); HPLC (purity): 97.1%; ¹H-NMR (CDCl₃) δ (ppm): 1.95-2.04 (m, 2 H), 2.32 (s, 3 H), 2.84-2.87 (m, 2 H), 3.69 (s, 3 H), 3.75-3.78 (m, 1 H), 4.06-4.13 (m, 2 H), 5.20 (d, 2 H), 6.78 (s, 1 H), 6.91 (s, 1 H), 7.22-7.32 (m, 4 H), 7.80 (d, 1 H), 8.09 (d, 1 H), and 8.60 (d, 1 H), 8.72 (s, 1 H); MS m/z: 400.3 (M⁺) |
| 11 | 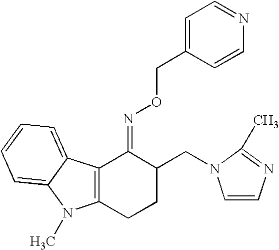 | R_{f} 0.54 (Chloroform: MeOH (9:1); HPLC (purity): 90.6%; ¹H-NMR (CDCl₃) δ (ppm): 1.95-2.04 (m, 2 H), 2.41 (s, 3 H), 2.86-2.88 (m, 2 H), 3.70 (s, 3 H), 3.83-3.87 (m, 2 H), 4.05-4.12 (m, 1 H), 5.34 (dd, 2 H), 6.83 (s, 1 H), 6.95 (s, 1 H), 7.20-7.22 (m, 1 H), 7.26-7.35 (m, 4 H), 8.02 (d, 1 H), and 8.60 (d, 2 H); MS m/z: 400.3 (M⁺) |
| 12 | 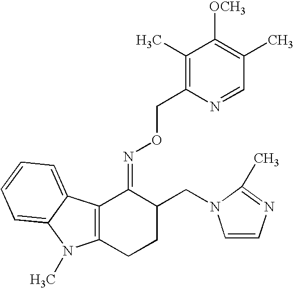 | R_{f} 0.57 (Chloroform: MeOH (9:1); HPLC (purity): 92.4%; ¹H-NMR (CDCl₃) δ (ppm): 1.95-2.04 (m, 2 H), 2.26 (s, 3 H), 2.27 (s, 3 H), 2.41 (s, 3 H), 2.83-2.86 (m, 2 H), 3.68 (s, 3 H), 3.72 (s, 3 H), 3.87-3.89 (m, 1 H), 4.14- 4.17 (m, 1 H), 4.19-4.23 (m, 1 H), 5.36 (d, 2 H), 6.80 (s, 1 H), 6.90 (s, 1 H), 7.22-7.31 (m, 3 H), 8.13 (d, 1 H), and 8.21 (s, 1 H); MS m/z: 458.4 (M⁺) |
| 13 | 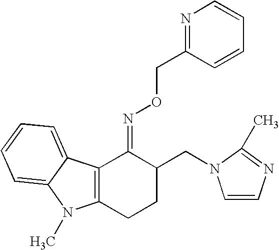 | R_{f} 0.50 (Chloroform: MeOH (9:1); HPLC (purity): 95.3%; ¹H-NMR (CDCl₃) δ (ppm): 1.95-2.04 (m, 2 H), 2.38 (s, 3 H), 2.85-2.88 (m, 2 H), 3.69 (s, 3 H), 3.78-3.82 (m, 1 H), 4.15-4.17 (m, 1 H), 4.19-4.23 (m, 1 H), 5.34 (s, 2 H), 6.85 (d, 1 H), 6.94 (d, 1 H), 7.19-7.21 (m, 3 H), 7.26-7.29 (m, 1 H), 7.48 (d, 1 H), 7.69 (d, 1 H), 8.07 (d, 1 H), and 8.60 (d, 1 H); MS m/z: 400.3 (M⁺) |
| 14 | 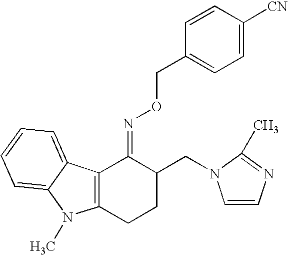 | R_{f} 0.48 (Chloroform: MeOH (9:1); HPLC (purity): 95.6%; ¹H-NMR (CDCl₃) δ (ppm): 1.95-2.04 (m, 2 H), 2.37 (s, 3 H), 2.85-2.89 (m, 2 H), 3.70 (s, 3 H), 3.78-3.84 (s, 1 H), 3.90-3.96 (m, 1 H), 4.08-4.13 (m, 1 H), 5.16 (dd, 2 H), 6.80 (s, 1 H), 6.93 (s, 1 H), 7.19-7.22 (t, 1 H), 7.27- 7.33 (m, 2 H), 7.54 (d, 2 H), 7.66 (d, 2 H), and 8.01 (d, 1 H); MS m/z: 424.2 (M⁺) |
| 15 | 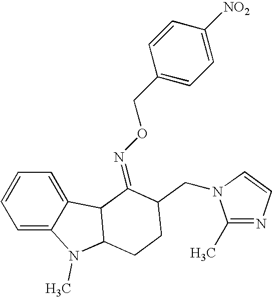 | R_{f} 0.65 (Chloroform: MeOH (9:1); HPLC (purity): 99.2%; m.p - 194-197° C.; ¹H-NMR (CDCl₃) δ (ppm): 1.98-2.02 (m, 2 H), 2.36 (s, 3 H), 2.86-2.89 (m, 2 H), 3.70 (s, 3 H), 3.76-3.80 (m, 1 H), 3.96-3.97 (m, 1 H), 4.09-4.14 (m, 1 H), 5.26-5.31 (dd, 2 H), 6.81 (s, 1 H), 6.91 (m, 1 H), 7.18-7.22 (m, 1 H), 7.26-7.32 (m, 2 H), 7.61 (d, 2 H), 8.01 (d, 1 H), and 8.22 (d, 2 H); MS m/z: 444.1 (M⁺) |
| 16 | 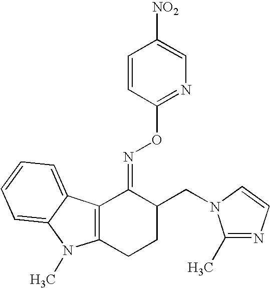 | R_{f} 0.53 (Chloroform: MeOH (9:1); HPLC (purity): 98.8%; ¹H-NMR (CDCl₃) δ (ppm): 1.97-2.04 (m, 2 H), 2.53 (s, 3 H), 2.97-2.99 (m, 2 H), 3.77 (s, 3 H), 3.97-4.01 (m, 1 H), 4.24-4.26 (m, 2 H), 6.88 (s, 1 H), 6.97 (s, 1 H), 7.34-7.39 (m, 3 H), 7.65 (d, 1 H), 8.13-8.15 (m, 1 H), 8.57-8.60 (dd, 1 H), and 9.17 (d, 1 H); MS m/z: 431.2 (M⁺) |
| 17 | 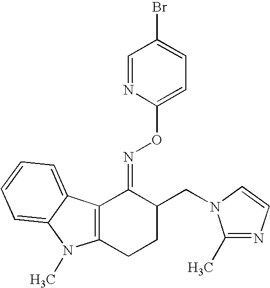 | R_{f} 0.7 (Chloroform: MeOH (9:1); HPLC (purity): 90.0% ¹H-NMR (CDCl₃) δ (ppm): 2.05-2.07 (m, 2 H), 2.54 (s, 3 H), 2.90-2.94 (m, 2 H), 3.74 (s, 3 H), 3.88-3.94 (m, 1 H), 4.24-4.29 (m, 2 H), 6.92 (d, 1 H), 6.95 (d, 1 H), 7.32-7.35 (m, 3 H), 7.45 (d, 1 H), 7.85 (d, 1 H), 8.15 (d, 1 H), and 8.32 (s, 1 H); MS m/z: 465.9 (M⁺) |
| 18 | 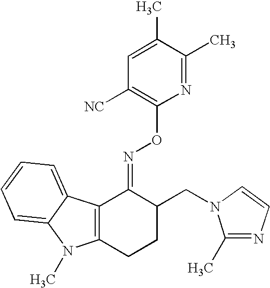 | R_{f} 0.67 (Chloroform: MeOH (9:1); HPLC (purity): 98.4%; ¹H-NMR (CDCl₃) δ (ppm): 1.95-2.04 (m, 2 H), 2.47 (s, 3 H), 2.53 (s, 3 H), 2.60 (s, 3 H), 2.88-2.89 (m, 2 H), 3.72 (s, 3 H), 3.93-3.97 (m, 1 H), 4.33-4.38 (m, 2 H), 6.87 (s, 1 H), 6.92 (s, 1 H), 7.09 (d, 1 H), 7.28-7.33 (m, 3 H), and 8.28-8.30 (t, 1 H); MS m/z: 439.3 (M⁺) |
| 19 | 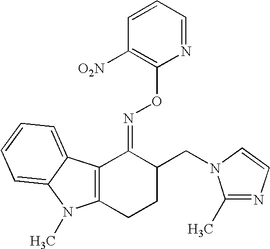 | R_{f} 0.62 (Chloroform: MeOH (9:1); HPLC (purity): 92.6%; ¹H-NMR (CDCl₃) δ (ppm): 1.98-2.01 (m, 2 H), 2.49 (s, 3 H), 2.92-2.93 (m, 2 H), 3.74 (s, 3 H), 3.99-4.00 (m, 1 H), 4.35-4.38 (m, 2 H), 6.94 (s, 1 H), 7.11 (s, 1 H), 7.19-7.22 (m, 1 H), 7.30-7.34 (m, 3 H), 8.21 (d, 1 H), 8.37 (dd, 1 H), and 8.61-8.63 (m, 1 H); MS m/z: 431.3 (M⁺) |
| 20 | 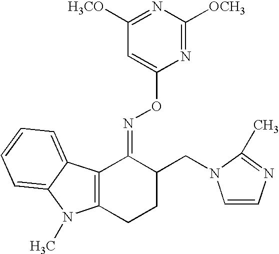 | R_{f} 0.55 (Chloroform: MeOH (9:1); HPLC (purity): 92.7%; ¹H-NMR (CDCl₃) δ (ppm): 2.07-2.12 (m, 2 H), 2.64 (s, 3 H), 2.92-2.96 (m, 2 H) 3.72 (s, 3 H), 3.75-3.77 (m, 1 H), 3.98 (s, 3 H), 3.99 (s, 3 H), 4.15-4.17 (m, 1 H), 4.24-4.28 (m, 1 H), 6.51 (s, 1 H), 6.97 (s, 2 H), 7.30-7.38 (m, 3 H), and 8.14-8.16 (m, 1 H); MS m/z: 447.4 (M⁺) |
| 21 | 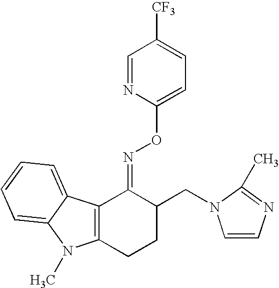 | R_{f} 0.80 (Chloroform: MeOH (9:1); HPLC (purity): 95.7% ¹H-NMR (CDCl₃) δ (ppm): 2.07-2.12 (m, 2 H), 2.54 (s, 3 H), 2.92-2.96 (m, 2 H) 3.75 (s, 3 H), 3.91-3.93 (m, 1 H), 4.23-4.26 (m, 2 H), 6.91 (s, 1 H), 6.95 (s, 1 H), 7.31- 7.37 (m, 3 H), 7.62-7.64 (d, 1 H), 8.00 (t, 1 H), 8.16 (t, 1 H), and 8.57 (s, IH); MS m/z: 454.2 (M⁺) |
| 22 | 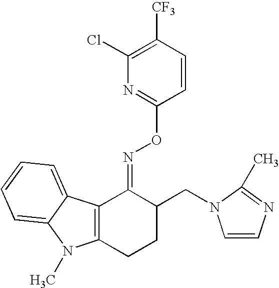 | R_{f} 0.70 (Chloroform: MeOH (9:1); HPLC (purity): 85.5%; ¹H-NMR (CDCl₃) δ (ppm): 2.06-2.13 (m, 2 H), 2.52 (s, 3 H), 2.90-2.94 (m, 2 H) 3.75 (s, 3 H), 3.91-3.95 (m, 1 H), 4.23-4.26 (m, 2 H) 6.92 (s, 1 H), 6.96 (d, 1 H), 7.31-7.38 (m, 3 H), 7.51-7.53 (m, 1 H), and 7.89 (s, 2 H); MS m/z: 488.2 (M⁺) |
| 23 | 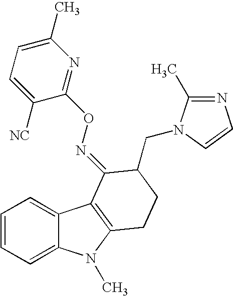 | R_{f} 0.32 (Chloroform: MeOH (9:1); HPLC (purity): 94.1%; ¹H-NMR (CDCl₃) δ (ppm): 1.95-1.96 (m, 1 H), 2.15- 2.17 (m, 2 H), 2.46 (s, 3 H), 2.65 (s, 3 H), 2.89-2.91 (m, 1 H) 3.71 (s, 3 H), 3.94-3.97 (m, 1 H), 4.32-4.35 (m, 2 H), 6.90 (s, 1 H), 6.99 (d, 1 H), 7.06 (d, 1 H), 7.30-7.34 (m, 3 H), and 7.85 (d, 1 H), 8.30 (t, 1 H); MS m/z: 424.9 (M⁺) |
| 24 | 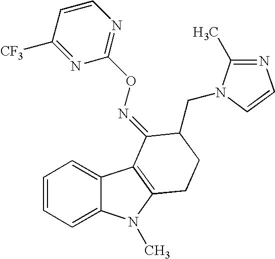 | R_{f} 0.35 (Chloroform: MeOH (9:1); HPLC (purity): 87.6%; ¹H-NMR (CDCl₃) δ (ppm): 2.09-2.14 (m, 2 H), 2.54 (s, 3 H), 2.93-2.96 (m, 2 H) 3.75 (s, 3 H), 3.90-3.96 (m, 1 H), 4.21-4.24 (s, 1 H), 4.28-4.33 (m, 1 H) 6.84 (s, 1 H), 6.99 (d, 1 H), 7.31-7.38 (m, 4 H), 8.31 (d, 1 H), and 8.90 (d, 1 H); MS m/z: 455.0 (M⁺) |

### Example 25

### Synthesis of (4E)-9-methyl-3-[(2-methyl-1H-imidazol-1-yl)methyl]-1,2,3,9-tetrahydro-4H-carbazol-4-one O-acetyloxime

To a solution of the oxime [prepared according to the procedure described in

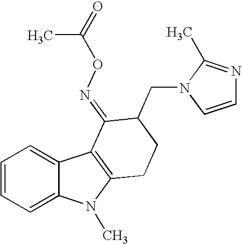

example 1] (0.1 g, 0.32 mmoles) in pyridine was added acetyl chloride (25.4 mg, 0.32 mmoles) at 0° C. (ice/water bath). The resulting slurry was stirred, until complete conversion (9:1, chloroform:methanol). The reaction mixture was subsequently poured into 25 ml of 5% aqueous sodium hydrogen carbonate solution. The organic layer was extracted with dichloromethane (20 ml), dried over anhydrous sodium sulfate and the organic solvent was evaporated under reduced pressure to yield a brown residue. The resulting residue was subjected to silica gel column chromatography, using a gradient of methanol in dichloromethane (0-10%), which in-turn yielded the desired product (72 mg, 63.4%), R_{f}=0.5 (9:1 chloroform:methanol); HPLC (purity): 87.0%; ¹H-NMR CDCl₃) δ (ppm): 8.19 (d, 1H), 7.34-7.29 (m, 3H), 6.93 (s, 1H), 6.83 (s, 1H), 4.16-4.11 (m, 1H), 3.95-3.88 (m, 1H), 3.73 (s, 3H), 2.94-2.90 (m, 2H), 2.47 (s, 3H), 2.28 (s, 3H), and 1.28-1.25 (m, 2H); MS m/z: 351.2 [M+1]
The Following Compounds have been Prepared by Using the Procedure Given in Example 25.

| | | |
|---|---|---|
| 26 | 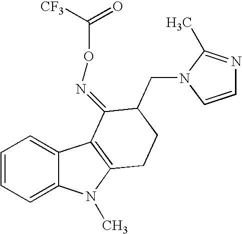 | R_{f} = 0.6 (9:1 CH₂Cl₂: CH₃OH); HPLC (purity): 96.5%; mp 148-150° C.; ¹H-NMR (CDCl₃) δ (ppm): 8.06 (d, 1 H), 7.74 (t, 1 H), 7.35-7.32 (m, 2 H), 7.95 (s, 1 H), 6.77 (s, 1 H), 4.15-4.09 (m, 2 H), 3.81 (m, 1 H), 3.71 (s, 3 H), 2.92 (m, 2 H), 2.05 (s, 3 H), and 2.09-1.52 (m, 2 H); IR (cm⁻¹) 3408.1, 1682.0, 1441.8, and 1284.7; MS m/z: 405.3 [M + 1] |
| 27 | 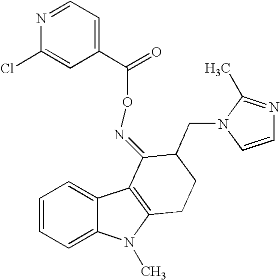 | R_{f} 0.55 (9:1 CH₂C₂: CH₃OH); HPLC (purity): 90.3%; mp 172-174° C.; ¹H-NMR (CDCl₃) δ (ppm): 8.90 (s, 1 H), 8.26 (d, 1 H), 8.18 (d, 1 H), 7.47 (d, 1 H), 7.34-7.26 (m, 3 H), 6.86 (m, 2 H), 4.19-4.06 (m, 3 H), 3.73 (s, 3 H), 2.92-2.88 (m, 2 H), 2.32 (s, 3 H), and 2.25-2.19 (m, 2 H); IR (cm⁻¹) 3391.8, 2925.2, 2851.4, 1731.9, 1586.1, 1477.8, 1457.9, and 1263.8; MS m/z: 448.1 [M + 1] |
| 28 | 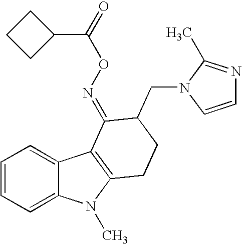 | R_{f} 0.6 (9:1 CH₂Cl₂: CH₃OH); HPLC (purity): 98.3%; ¹H-NMR (CDCl₃) δ (ppm): 8.21 (t, 1 H), 7.34-7.26 (m, 3 H), 7.01 (s, 1 H), 6.86 (s, 1 H), 4.38 (m, 1 H), 4.13-3.91 (m, 2 H), 3.72 (s, 3 H), 3.62- 3.59 (m, 1 H), 3.21-3.15 (m, 2 H), 2.92-2.88 (m, 2 H), 2.35 (s, 3 H), and 2.35-1.92 (m, 6 H); MS m/z: 391.2 [M + 1] |
| 29 | 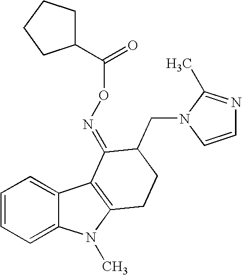 | R_{f} = 0.65 (9:1 CHCl₃: CH₃OH); HPLC (purity): 97.0%; ¹H-NMR (CDCl₃) δ (ppm): 8.23 (d, 1 H), 7.34-7.24 (m, 3 H), 7.00 (s, 1 H), 6.85 (s, 1 H), 4.14 (dd, 1 H), 3.92-3.88 (m, 2 H), 3.72 (s, 3 H), 2.92- 2.76 (m, 3 H), 2.74-2.56 (m, 2 H), 2.52 (s, 3 H), and 1.97-1.82 (m, 8 H); MS m/z: 405.4 [M + 1] |
| 30 | 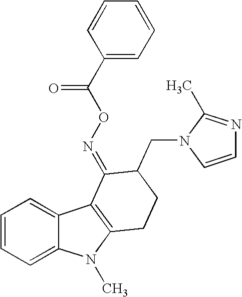 | R_{f} = 0.70 (9:1 CHCl₃: CH₃OH); HPLC (purity): 94.9%; mp 179-185° C.; ¹H-NMR (CDCl₃) δ (ppm): 8.34 (d, 1 H), 8.04 (d, 2 H), 7.62 (t, 1 H), 7.52-7.48 (m, 2 H), 7.32-7.28 (m, 3 H), 6.94 (s, 1 H), 6.86 (s, 1 H), 4.22-4.17 (m, 1 H), 4.04-3.95 (m, 2 H), 3.71 (s, 3 H), 2.98-2.88 (m, 2 H), 2.54 (s, 3 H), 2.22-2.19 (m, 1 H), and 2.03-2.01 (s, 1 H); IR (cm⁻¹) 3248.8, 2933.0, 2851.4, 1726.6, 1570.1, 1478.0, 1449.5, and 1257.5; MS m/z: 413.3 [M + 1] |
| 31 | 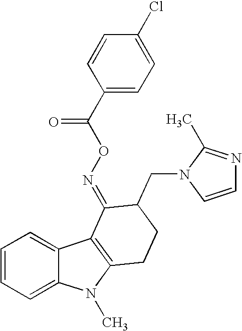 | R_{f} = 0.76 (9:1 CHCl₃: CH₃OH); HPLC (purity): 99.4%; mp 206-212° C.; ¹H-NMR (CDCl₃) δ (ppm): 8.32 (d, 1 H), 7.95 (d, 2 H), 7.47 (d, 2 H), 7.52-7.48 (m, 2 H), 7.32-7.30 (m, 3 H), 6.90 (s, 1 H), 6.82 (s, 1 H), 4.19-4.15 (m, 1 H), 4.02-3.95 (m, 2 H), 3.72 (s, 3 H), 2.98-2.83 (m, 2 H), 2.34 (s, 3 H), and 2.22-2.07 (m, 2 H); IR (cm⁻¹) 3430.0, 2933.7, 2851.4, 1727.4, 1569.4.1, 1476.3, 1399.2, and 1254.9; MS m/z: 447.3 [M + 1] |
| 32 | 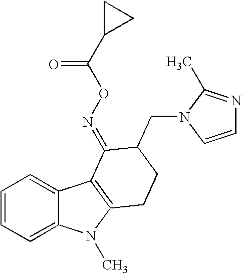 | R_{f} = 0.6 (9:1 CHCl₃: CH₃OH); HPLC (purity): 97.2%; ¹H-NMR (CDCl₃) δ (ppm): 8.22 (d, 1 H), 7.37-7.24 (m, 3 H), 6.99 (s, 1 H), 6.85 (s, 1 H), 4.19-4.15 (m, 1 H), 3.94-3.90 (m, 2 H), 3.72 (s, 3 H), 3.62-3.59 (m, 1 H), 3.21-3.15 (m, 2 H), 2.93- 2.89 (m, 2 H), 2.54 (s, 3 H), 2.09-2.04 (m, 2 H), 1.75-1.73 (m, 1 H), and 1.69-1.60 (m, 2 H); IR (cm⁻¹) 3434.0, 2927.7, 1743.5, 1589.4, 1478.5, 1381.2, and 1281.6; MS m/z: 377.3 [M + 1] |
| 33 | 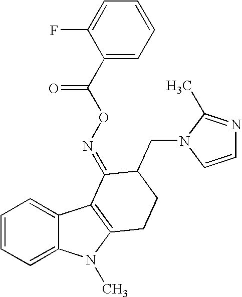 | R_{f} 0.70 (9:1 CHCl₃: CH₃OH); HPLC (purity): 99.4%; mp 172-175° C.; ¹H-NMR (CDCl₃) δ (ppm): 8.32 (d, 1 H), 8.03 (t, 1 H), 7.95 (t, 1 H), 7.57 (d, 1 H), 7.44-7.42 (m, 1 H), 7.32-7.01 (m, 4 H), 6.93 (s, 1 H), 4.26-4.21 (m, 1 H), 4.14-4.12 (m, 1 H), 4.01-3.95 (m, 1 H), 3.70 (s, 3 H), 2.93- 2.82 (m, 2 H), 2.48 (s, 3 H), and 2.18-1.94 (m, 2 H); IR (cm⁻¹) 3419.5, 2932.8, 1722.1, 1611.7 1571.6, 1478.0, 1403.9, and 1288.9; MS m/z: 431.3 [M + 1] |
| 34 | 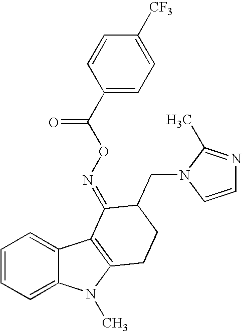 | R_{f} = 0.62 (9:1 CHCl₃: CH₃OH); HPLC (purity): 100%; mp 200-203° C.; ¹H-NMR (CDCl₃) δ (ppm): 8.30 (d, 1 H), 8.19 (d, 2 H), 8.06 (d, 2 H), 7.76 (d, 1 H), 7.70 (d, 1 H), 7.32-7.28 (m, 1 H), 7.00 (s, 1 H), 6.83 (s, 1 H), 4.21-4.06 (m, 3 H), 3.73 (s, 3 H), 3.70 (s, 3 H), 3.01-2.96 (m, 2 H), 2.48 (s, 3 H), 2.29-2.16 (m, 1 H) and 2.14-2.12 (m, 1 H); IR (cm⁻¹) 3435.7, 2935.8, 1733.0, 1584.9, 1570.2, 1476.6, 1408.4, and 1262.1; MS m/z: 481.2 [M + 1] |
| 35 | 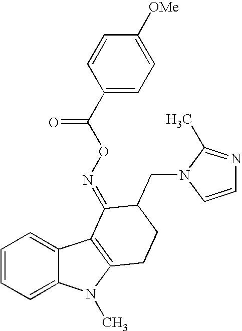 | R_{f} 0.80 (9:1 CHCl₃: CH₃OH); HPLC (purity): 99.6%; mp 200-203° C.; ¹H-NMR (CDCl₃) δ (ppm): 8.34 (d, 1 H), 8.01 (d, 2 H), 7.32-7.28 (m, 3 H), 6.97 (d, 2 H), 6.91 (s, 1 H), 6.85 (s, 1 H), 4.21- 4.04 (m, 2 H), 3.98-3.92 (m, 2 H), 3.90 (s, 3 H), 3.70 (s, 3 H), 2.92-2.87 (m, 2 H), 2.01 (m, 1 H) and 1.94 (m, 1 H); IR (cm⁻¹) 3409.5, 2931.8, 1717.0, 1603.0, 1571.6, 1476.0, 1401.0, and 1254.1; MS m/z: 443.2 [M + 1] |
| 36 | 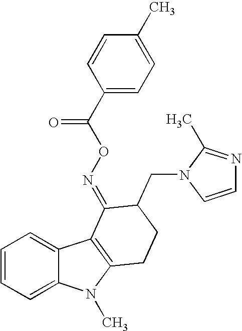 | R_{f} 0.70 (9:1 CHCl₃: CH₃OH); HPLC (purity): 96.8%; mp 207-212° C.; ¹H-NMR (CDCl₃) δ (ppm): 8.32 (d, 1 H), 7.92 (d, 2 H), 7.31-7.26 (m, 5 H), 6.96 (s, 1 H), 6.89 (s, 1 H), 4.26-4.21 (m, 1 H), 4.21-4.17 (m, 1 H), 4.05-3.98 (m, 1 H), 3.70 (s, 3 H), 2.98-2.89 (m, 2 H), 2.45 (s, 3 H), 2.42 (s, 3 H), 2.38-2.24 (m, 1 H), and 2.18-2.03 (m, 1 H); IR (cm⁻¹) 3418.1, 2925.0, 1720.8, 1609.1 1570.9, 1477.2, 1401.6, and 1259.8; MS m/z: 427.2 [M + 1] |
| 37 | 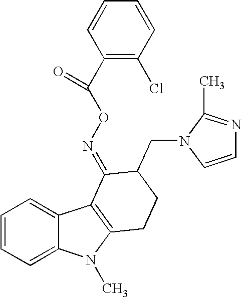 | R_{f} = 0.58 (9:1 CHCl₃: CH₃OH); HPLC (purity): 97.2%; mp 111-116° C.; ¹H-NMR (CDCl₃) δ (ppm): 8.26 (d, 1 H), 7.76 (d, 1 H), 7.51-7.41 (m, 2 H), 7.40-7.38 (m, 1 H), 7.33-7.26 (m, 3 H), 6.89 (s, 1 H), 6.80 (s, 1 H), 4.22-4.17 (m, 1 H), 4.01 (m, 1 H), 3.93-3.87 (m, 1 H), 3.73 (s, 3 H), 2.93-2.88 (m, 2 H), 2.38 (s, 3 H), and 2.23-2.04 (m, 2 H); IR (cm⁻¹) 3412.1, 2926.7, 1742.2, 1589.6, 1570.0, 1477.5, 1435.2, and 1272.9; MS m/z: 447.1 [M + 1] |
| 38 | 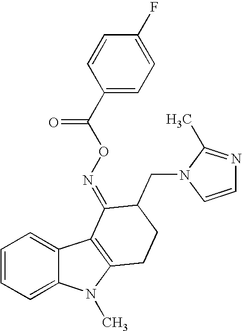 | R_{f} 0.65 (9:1 CHCl₃: CH₃OH); HPLC (purity): 97.6%; mp 192-200° C.; ¹H-NMR (CDCl₃) δ (ppm): 8.32 (t, 1 H), 8.07-8.03 (m, 2 H), 7.76 (d, 1 H), 7.51-7.41 (m, 2 H), 7.33-7.28 (m, 3 H), 7.19- 7.15 (m, 2 H), 6.90 (s, 1 H), 6.83 (s, 1 H), 4.20- 4.16 (m, 1 H), 4.04-3.97 (m, 2 H), 3.73 (s, 3 H), 2.98-2.88 (m, 2 H), 2.34 (s, 3 H), 2.23-2.17 (m, 2 H), and 2.07-2.03 (m, 1 H); IR (cm⁻¹) 3436.2, 2934.4, 1729.2, 1603.2, 1570.1, 1477.0, 1404.1, and 1256.8; MS m/z: 431.3 [M + 1] |
| 39 | 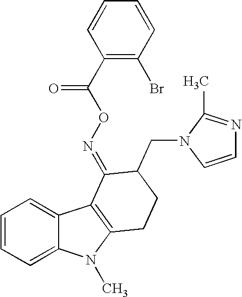 | R_{f} 0.58 (9:1 CHCl₃: CH₃OH); HPLC (purity): 98.0%; mp 108-112° C.; ¹H-NMR (CDCl₃) δ (ppm): 8.25 (d, 1 H), 7.70-7.60 (m, 2 H), 7.45- 7.24 (m, 5 H), 6.98 (s, 1 H), 6.80 (s, 1 H), 4.23- 4.20 (m, 1 H), 4.03-3.94 (m, 2 H), 3.74 (s, 3 H), 2.98-2.88 (m, 2 H), 2.31 (s, 3 H), and 2.23-2.02 (m, 2 H); IR (cm⁻¹) 3435.8, 2924.1, 1728.2, 1570.5, 1476.3, 1303.1, and 1245.1; MS m/z: 491.4 [M + 1] |
| 40 | 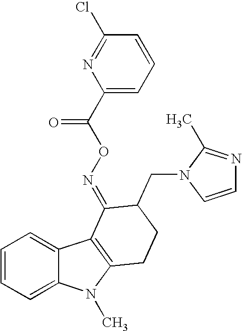 | R_{f} = 0.64 (9:1 CHCl₃: CH₃OH); HPLC (purity): 96.5%; mp 170-175° C.; ¹H-NMR (CDCl₃) δ (ppm): 8.34 (d, 1 H), 8.20 (d, 1 H), 7.87 (t, 1 H), 7.57 (d, 1 H), 7.37-7.26 (m, 4 H), 6.95 (s, 1 H), 4.28-4.25 (m, 2 H), 3.95-3.88 (m, 2 H), 3.75 (s, 3 H), 2.95-2.88 (m, 2 H), 2.50 (s, 3 H), and 2.14- 1.97 (m, 2 H); IR (cm⁻¹) 3430.1, 2925.4, 1728.5, 1570.4, 1477.1, 1303.1, and 1245.1; MS m/z: 448.1 [M + 1] |
| 41 | 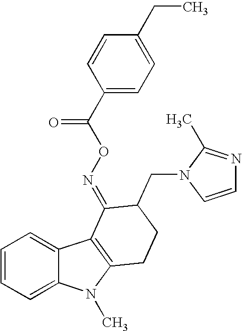 | R_{f} = 0.50 (9:1 CHCl₃: CH₃OH); HPLC (purity): 97.4%; mp 175-178° C.; ¹H-NMR (CDCl₃) δ (ppm): 8.34 (d, 1 H), 7.99 (m, 2 H), 7.33-7.26 (m, 5 H), 6.96 (s, 1 H), 6.87 (s, 1 H), 4.24-4.19 (m, 1 H), 4.07 (s, 1 H), 3.98-3.92 (m, 1 H), 3.72 (s, 3 H), 2.92-2.87 (m, 2 H), 2.77-2.70 (m, 2 H), 2.38 (s, 3 H), 2.17-1.98 (m, 2 H), and 1.28 (t, 3 H); IR (cm⁻¹) 3435.7, 2929.4, 1722.5, 1571.6, 1476.3, 1260.3, and 1178.6; MS m/z: 441.3 [M + 1] |
| 42 | 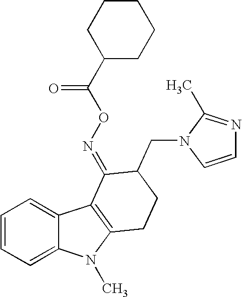 | R_{f} = 0.73 (9:1 CHCl₃: CH₃OH); HPLC (purity): 97.3%; mp 98-106° C.; ¹H-NMR (CDCl₃) δ (ppm): 8.25 (d, 1 H), 7.31-7.26 (m, 3 H), 6.97 (s, 1 H), 6.85 (s, 1 H), 4.21-4.13 (m, 1 H), 3.93-3.88 (m, 2 H), 3.71 (s, 3 H), 2.90-2.86 (m, 2 H), 2.48 (s, 3 H), and 1.98-1.25 (m, 13 H); IR (cm⁻¹) 3434.6, 2932.0, 1747.5, 1587.6, 1476.2, 1279.1, and 1159.2; MS m/z: 419.2 [M + 1] |
| 43 | 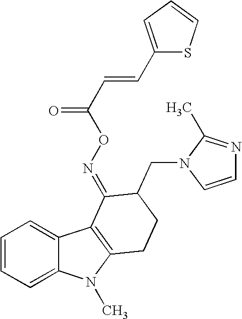 | R_{f} = 0.68 (9:1 CHCl₃: CH₃OH); HPLC (purity): 98.9%; mp 185-189° C.; ¹H-NMR (CDCl₃) δ (ppm): 8.28 (t, 1 H), 7.94 (d, 1 H), 7.42 (d, 1 H), 7.32-7.26 (m, 4 H), 7.08 (t, 1 H), 6.96 (s, 1 H), 6.88 (s, 1 H), 6.44 (s, 1 H), 6.40 (s, 1 H), 4.21-4.16 (m, 1 H), 3.98-3.88 (m, 2 H), 3.72 (s, 3 H), 2.93- 2.90 (m, 2 H), 2.50 (s, 3 H), and 2.14-2.05 (m, 2 H); IR (cm⁻¹) 3422.4, 2929.4, 1712.6, 1624.4, 1475.4, 1274.1, and 1133.4; MS m/z: 445.1 [M + 1] |
| 44 | 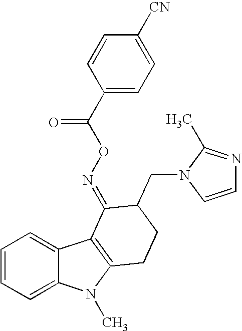 | R_{f} = 0.65 (9:1 CHCl₃: CH₃OH); HPLC (purity) 97.4%; mp 178-180° C.; ¹H-NMR (CDCl₃) δ (ppm): 8.28 (t, 1 H), 7.94 (d, 1 H), 7.42 (d, 1 H), 7.32-7.26 (m, 4 H), 7.08 (t, 1 H), 6.96 (s, 1 H), 6.88 (s, 1 H), 4.21-4.16 (m), 3.98-3.88 (m, 2 H), 3.74 (s, 3 H), 2.93-2.90 (m, 2 H), 2.31 (s, 3 H), 2.24-2.14 (m, 1 H), and 2.08-1.89 (1 H); IR (cm⁻¹) 3435.8, 2930.4, 1731.6, 1569.1, 1478.2, and 1265.5; MS m/z: 438.3 (M⁺) |
| 45 | 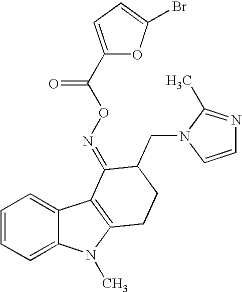 | R_{f} = 0.55 (9:1 CHCl₃: CH₃OH); HPLC (purity) 98.3%; mp 180187° C.; ¹H-NMR (CDCl₃) δ (ppm): 8.29 (m, 1 H), 7.31-7.26 (m, 4 H), 7.04 (s, 1 H), 6.97 (s, 1 H), 6.55 (s, 1 H), 4.26-4.21 (m, 1 H), 4.11-4.09 (m, 1 H), 3.96-3.90 (m, 1 H), 3.73 (s, 3 H), 2.95-2.88 (m, 2 H), 2.53 (s, 3 H), and 2.15- 2.08 (m, 2 H); IR (cm⁻¹) 3423.9, 2929.1, 1721.6, 1571.2, 1465.8, and 1287.2; MS m/z: 483.2 [M + 1] |
| 46 | 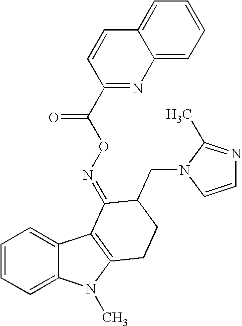 | R_{f} = 0.72 (9:1 CHCl₃: CH₃OH); HPLC (purity) 91.6%; mp 170-178° C.; ¹H-NMR (CDCl₃) δ (ppm): 8.40-8.30 (m, 4 H), 7.93 (d, 1 H), 7.84 (t, 1 H), 7.69 (t, 1 H), 7.42-7.26 (m, 4 H), 6.98 (s, 1 H), 4.40-4.37 (m, 2 H), 3.98-3.92 (m, 1 H), 3.75 (s, 3 H), 3.01-2.95 (m, 2 H), 2.49 (s, 3 H), and 2.19- 2.01 (m, 2 H); IR (cm⁻¹) 3420.0, 2930.9, 1721.9, 1570.7, 1477.7, and 1313.2; MS m/z: 464.3 [M + 1] |
| 47 | 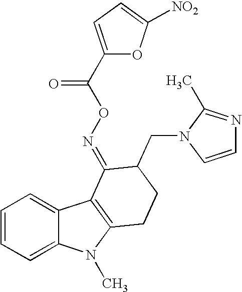 | R_{f} 0.65 (9:1 CHCl₃: CH₃OH); HPLC (purity) 94.9%; mp 186-190° C.; ¹H-NMR (CDCl₃) δ (ppm): 8.03 (d, 1 H), 7.88 (d, 1 H), 7.70 (d, 1 H), 7.57 (d, 1 H), 7.28-7.21 (m, 2 H), 7.08 (s, 1 H), 6.62 (s, 1 H), 4.12-4.09 (m, 3 H), 3.76 (s, 3 H), 3.08-3.03 (m, 2 H), 2.53 (s, 3 H), and 2.15-2.08 (m, 2 H); MS m/z: 448.1 [M + 1] |
| 48 | 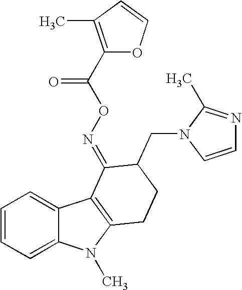 | R_{f} = 0.50 (9:1 CHCl₃: CH₃OH); HPLC (purity) 97.6%; mp 165-170° C.; ¹H-NMR (CDCl₃) δ (ppm): 8.33 (d, 1 H), 7.46 (s, 1 H), 7.31-7.26 (m, 3 H), 7.00 (d, 2 H), 6.97 (s, 1 H), 4.30-4.26 (m, 1 H), 4.12-4.06 (m, 1 H), 3.90-3.87 (m, 1 H), 3.73 (s, 3 H), 2.92-2.88 (m, 2 H), 2.53 (s, 3 H), 2.47 (s, 3 H), and 2.10-1.97 (m, 2 H); MS m/z: 417.2 [M + 1] |
| 49 | 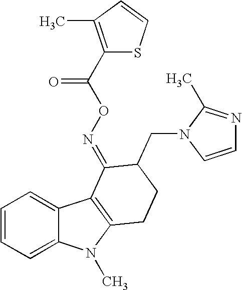 | R_{f} = 0.48 (9:1 CHCl₃: CH₃OH); HPLC (purity): 94.1%; mp 177-180° C.; ¹H-NMR (CDCl₃) δ (ppm): 8.34-8.32 (m, 1 H), 7.44 (t, 1 H), 7.34-7.26 (m, 3 H), 7.00 (d, 2 H), 6.95 (s, 1 H), 4.25-4.20 (m, 1 H), 4.09-4.02 (m, 1 H), 3.97-3.90 (m, 1 H), 3.71 (s, 3 H), 2.89-2.81 (m, 2 H), 2.68 (s, 3 H), 2.43 (s, 3 H), and 2.14-1.98 (m, 2 H); IR (cm⁻¹) 3420.6, 2928.3, 1721.2, 1571.4, 1480.8, 1251.2, and 1061.4; MS m/z: 433.3 [M + 1] |
| 50 | 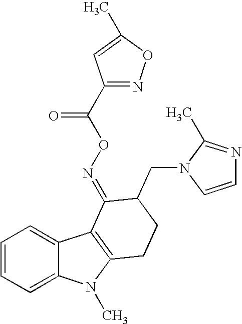 | R_{f} = 0.60 (9:1 CHCl₃: CH₃OH); HPLC (purity): 94.7%; mp 154-157° C.; ¹H-NMR (CDCl₃) δ (ppm): 8.30 (d, 1 H), 7.44 (t, 1 H), 7.34-7.29 (m, 3 H), 7.13 (s, 1 H), 6.94 (s, 1 H), 6.54 (s, 1 H), 4.27- 3.94 (m, 2 H), 3.93-3.89 (m, 1 H), 3.74 (s, 3 H), 2.94-2.90 (m, 2 H), 2.54 (s, 3 H), 2.47 (s, 3 H), and 2.12-1.95 (m, 2 H); IR (cm⁻¹) 3400.2, 2933.6, 1753.4, 1568.3, 1479.6, 1274.6, and 1184.3; MS m/z: 418.2 [M + 1] |
| 51 | 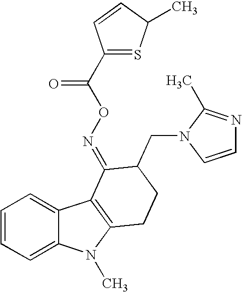 | R_{f} 0.66 (9:1 CHCl₃: CH₃OH); HPLC (purity): 96.7%; ¹H-NMR (CDCl₃) δ (ppm): 8.31 (d, 1 H), 7.73 (d, 1 H), 7.35-7.28 (m, 4 H), 6.95 (d, 1 H), 6.84 (d, 2 H), 4.21-4.04 (m, 1 H), 3.96-3.71 (m, 2 H), 3.71 (s, 3 H), 2.89-2.83 (m, 2 H), 2.57 (s, 3 H), 2.42 (s, 3 H), and 2.12-1.95 (m, 2 H); IR (cm⁻¹), 3420.3, 2956.7, 1722.3, 1584.3, 1457.9, 1246.8, and 1072.3; MS m/z: 433.3 [M + 1] |
| 52 | 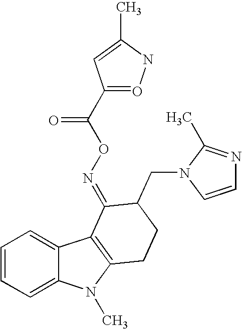 | R_{f} = 0.60 (9:1 CHCl₃: CH₃OH); HPLC (purity): 94.7%; mp 182-191° C.; ¹H-NMR (CDCl₃) δ (ppm): 8.78 (s, 1 H), 8.24 (d, 1 H), 7.36-7.29 (m, 3 H), 6.93 (s, 1 H), 6.81 (s, 1 H), 4.14-4.06 (m, 1 H), 4.04-3.97 (m, 1 H), 3.74 (s, 3 H), 2.96-2.89 (m, 2 H), 2.56 (s, 3 H), 2.38 (s, 3 H), and 2.17-2.08 (m, 2 H); IR (cm⁻¹) 3426.2, 2923.9, 1752.2, 1586.4, and 1479.5, 1287.5, 1135.3; MS m/z: 418.2 [M + 1] |
| 53 | 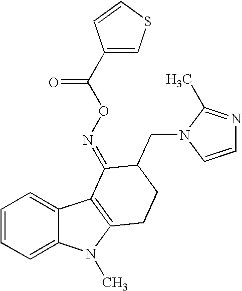 | R_{f} = 0.60 (9:1 CHCl₃: CH₃OH); HPLC (purity): 91.0%; mp 171-175° C.; ¹H-NMR (CDCl₃) δ (ppm): 8.32 (t, 1 H), 8.14 (d, 1 H), 7.56 (d, 1 H), 7.39-7.29 (m, 4 H), 6.91 (s, 1 H), 6.84 (d, 1 H), 4.20-4.15 (m, 1 H), 4.02-3.95 (m, 2 H), 3.72 (s, 3 H), 2.92-2.86 (m, 2 H), 2.38 (s, 3 H), and 2.12- 2.04 (m, 2 H); IR (cm⁻¹) 3431.1, 2921.9, 1729.4, 1569.3, 1479.2, 1283.3, and 1193.4; MS m/z: 419.2 [M + 1] |
| 54 | 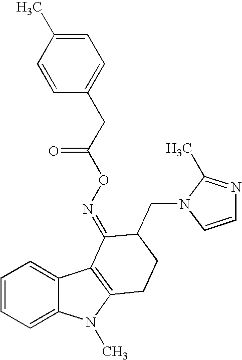 | R_{f} = 0.57 (9:1 CHCl₃: CH₃OH); HPLC (purity): 90.7%; ¹H-NMR (CDCl₃) δ (ppm): 8.20 (d, 1 H), 7.31-7.22 (m, 4 H), 7.14 (d, 2 H), 6.93 (d, 2 H), 6.65 (s, 1 H), 4.01-3.97 (m, 1 H), 3.84-3.80 (m, 4 H), 3.70 (s, 3 H), 2.92-2.86 (m, 2 H), 2.41 (s, 3 H), 2.32 (s, 3 H), and 2.12-2.04 (m, 2 H); IR (cm⁻¹) 3431.1, 2923.5, 1752.4, 1589.9, 1467.6, 1278.4, and 1119.6; MS m/z: 441.3 [M + 1] |
| 55 | 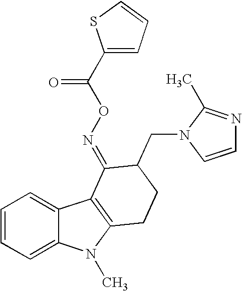 | R_{f} = 0.57 (9:1 CHCl₃: CH₃OH); HPLC (purity): 96.9%; ¹H-NMR (CDCl₃) δ (ppm): 8.32 (d, 1 H), 7.93 (s, 1 H), 7.63 (s, 1 H), 7.32-7.29 (m, 4 H), 7.18 (t, 1 H), 6.96 (d, 1 H), 4.27-4.20 (m, 1 H), 4.14- 4.07 (m, 1 H), 3.97-3.90 (m, 1 H), 3.72 (s, 3 H), 2.93-2.85 (m, 2 H), 2.42 (s, 3 H), and 2.12-2.04 (m, 2 H); IR (cm⁻¹) 3401.8, 2955.8, 1752.3, 1588.2, 1467.6, 1276.6, and 1119.8; MS m/z: 419.1 [M + 1] |
| 56 | 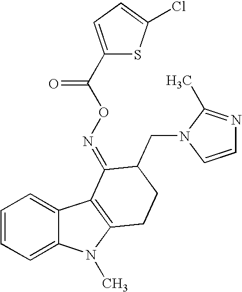 | R_{f} = 0.50 (9:1 CHCl₃: CH₃OH); HPLC (purity) 97.8%; mp 140-144° C.; ¹H-NMR (CDCl₃) δ (ppm): 8.29 (m, 1 H), 7.67 (d, 1 H), 7.31-7.26 (m, 3 H), 7.01-6.97 (m, 3 H), 4.16-4.12 (m, 1 H), 4.02- 3.98 (m, 2 H), 3.73 (s, 3 H), 2.96-2.84 (m, 2 H), 2.43 (s, 3 H), and 2.19-2.04 (m, 2 H); MS m/z: 453 [M + 1] |
| 57 | 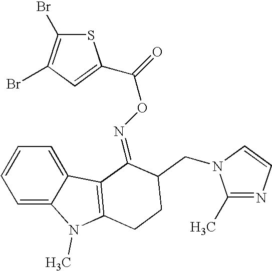 | R_{f} 0.74 (Chloroform: MeOH (9:1); HPLC (purity): 98.7%; ¹H-NMR (CDCl₃) δ (ppm): 2.07- 2.12 (m, 2 H), 2.40 (s, 3 H), 2.84-2.92 (m, 2 H) 3.72 (s, 3 H), 3.98-4.00 (m, 2 H), 4.15-4.17 (m, 1 H) 6.88 (s, 1 H), 6.94 (d, 1 H), 7.64 (s, 1 H), 7.29- 7.33 (m, 3 H), and 8.26 (s, 1 H); MS m/z: 576.8 (M⁺) |
| 58 | 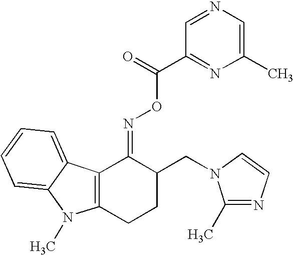 | R_{f} 0.56 (Chloroform: MeOH (9:1); HPLC (purity): 95.4%; m.p - 183-190° C.; ¹H-NMR (CDCl₃) δ (ppm): 1.95-1.98 (m, 2 H), 2.48 (s, 3 H), 2.71 (s, 3 H), 2.93-2.96 (m, 2 H), 3.74 (s, 3 H), 3.92-3.95 (m, 1 H), 4.27-4.30 (m, 2 H), 6.92 (s, 1 H), 7.13 (s, 1 H), 7.31-7.34 (m, 3 H), 8.34- 8.36 (m, 1 H), 8.57 (s, 1 H), and 9.29 (d, 1 H); MS m/z: 429.2 (M⁺) |
| 59 | 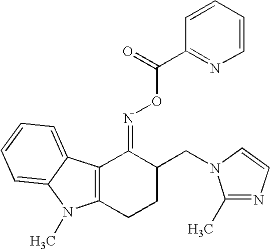 | R_{f} 0.60 (Chloroform: MeOH (9:1); HPLC (purity): 96.0%; m.p - 162-165° C.; ¹H-NMR (CDCl₃) δ (ppm): 1.95-1.98 (m, 2 H), 2.48 (s, 3 H), 2.94-2.96 (m, 2 H), 3.74 (s, 3 H), 3.88-3.91 (m, 1 H), 4.30-4.35 (m, 2 H), 6.94 (s, 1 H), 6.97 (m, 1 H), 7.24-7.34 (m, 3 H), 7.53-7.56 (m, 1 H), 7.91 (t, 1 H), 8.24 (d, 1 H), 8.36 (t, 1 H), and 8.74 (d, 1 H); MS m/z: 414.2 (M⁺) |
| 60 | 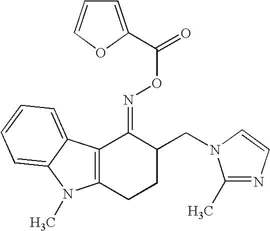 | R_{f} 0.58 (Chloroform: MeOH (9:1); HPLC (purity): 96.1%; m.p - 168-171° C.; ¹H-NMR (CDCl₃) δ (ppm): 1.98-2.01 (m, 2 H), 2.52 (s, 3 H), 2.93-2.95 (m, 2 H), 3.72 (s, 3 H), 3.98-4.00 (m, 1 H), 4.20-4.22 (m, 2 H), 6.59-6.60 (q, 1 H), 6.97 (s, 2 H), 7.27-7.33 (m, 4 H), 7.64 (s, 1 H), and 8.28 (d, 1 H); MS m/z: 403.2 (M⁺) |
| 61 | 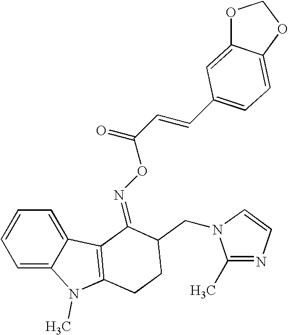 | R_{f} 0.54 (Chloroform: MeOH (9:1), HPLC (purity): 87.8%; m.p - 139-144° C.; ¹H-NMR (CDCl₃) δ (ppm): 2.03-2.08 (m, 2 H), 2.55 (s, 3 H), 2.91-2.94 (m, 2 H), 3.72 (s, 3 H), 3.97-3.99 (m, 2 H), 4.17-4.19 (m, 1 H), 6.03 (s, 2 H), 6.38 (d, 1 H), 6.86 (s, 1 H), 6.91 (s, 1 H), 7.02-7.06 (m, 3 H), 7.28-7.33 (m, 3 H), and 7.74 (d, 1 H), 8.27 (d, 1 H); MS m/z: 483.2 (M⁺) |
| 62 | 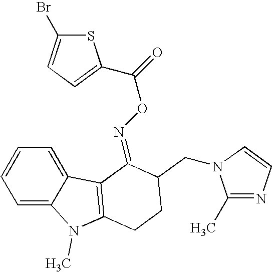 | R_{f} 0.6 (Chloroform: MeOH (9:1); HPLC (purity): 91.4%; ¹H-NMR (CDCl₃) δ (ppm): 1.95-2.04 (m, 1 H), 2.12-2.17 (m, 1 H), 2.42 (s, 3 H), 2.84-2.92 (m, 2 H), 3.72 (s, 3 H), 3.98-4.02 (m, 2 H), 4.16- 4.17 (m, 1 H), 6.92 (d, 1 H), 6.94 (d, 1 H), 7.14 (d, 1 H), 7.28-7.35 (m, 3 H), 7.64 (d, 1 H), and 8.27- 8.29 (m, 1 H); MS m/z: 497.1 (M⁺) |
| 63 | 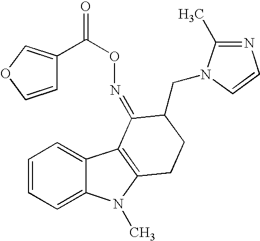 | R_{f} 0.50 (Chloroform: MeOH (9:1); HPLC (purity): 96.4%; m.p 158-164° C.; ¹H-NMR (CDCl₃) δ (ppm): 2.04-2.09 (m, 2 H), 2.44 (s, 3 H), 2.86-2.89 (m, 2 H), 3.72 (s, 3 H), 3.94-3.99 (m, 2 H), 4.14-4.17 (m, 1 H), 6.77 (s, 1 H), 6.85 (s, 1 H), 6.99 (s, 1 H), 7.27-7.32 (m, 3 H), 7.49 (t, 1 H), 8.06 (m, 1 H), and 8.29 (d, 1 H); MS m/z: 403.3 (M⁺) |

### Example 64

### Synthesis of 4-(hydroxyamino)-9-methyl-3-[(2-methyl-1H-imidazol-1-yl)methyl]-2,3,4,9-tetrahydro-1H-carbazole

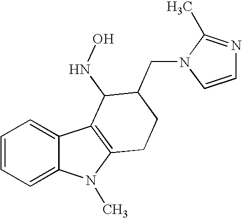

To a solution of the oxime [prepared according to the procedure described in example 1] (0.5 g, 1.62 mmoles) in dry methanol (3 ml) was added borane pyridine complex (348 mg, 3.75 mmoles) at 0° C. (ice/water bath). The resulting slurry was stirred for 12 hours at room temperature. Subsequently 6N hydrochloric acid (3 ml) was added to the reaction mixture and the resulting solution was stirred for another 6 hours. The reaction was then neutralized with 2N sodium hydroxide, to pH 9.0. The organic layer was extracted with dichloromethane (20 ml), dried over anhydrous sodium sulfate and evaporated at reduced pressure to yield a residue. The resulting residue was subjected to silica gel column chromatography, using a gradient of methanol in dichloromethane (0-10%) which gave the desired product (140 mg, 28.1%); R_{f} 0.5 (9:1 dichloromethane:methanol); HPLC (purity): 92.5%; mp 140-144° C.; ¹H-NMR (CDCl₃) δ (ppm): 8.29 (d, 1H), 7.30-7.23 (m, 3H), 7.17 (t, 1H), 6.96 (t, 1H), 6.81 (bs, 1H), 4.21-4.14 (m, 3H), 3.70 (s, 3H), 2.92 (m, 2H), 2.34 (s, 3H), and 2.09-2.04 (m, 2H); IR (cm⁻¹) 3215.1, 2925.7, 2852.7, 1635.7, 1476.8, 1419.1, and 1279.3; MS m/z: 310.4[M+1]

### Example 65

### Synthesis of (4E)-9-methyl-3-[(2-methyl-1H-imidazol-1-yl)methyl]-1,2,3,9-tetrahydro-4H-carbazol-4-one O-3-cyanobenzoyloxime

To the slurry of the oxime [prepared according to the procedure described in

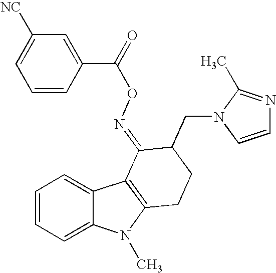

example 1] (0.4 g, 1.29 mmoles) in dry DMF (5 ml) were added EDCI (247 mg, 1.29 mmoles), HOBT (174 mg, 1.29 mmoles), and 3-cyano benzoic acid (189 mg, 1.29 mmoles). The resulting slurry was stirred for 48 hours at room temperature. Subsequently the reaction mixture was poured into 25 ml of saturated aqueous sodium chloride solution, and the organic layer was extracted with dichloromethane (20 ml), dried over anhydrous sodium sulfate and evaporated at reduced pressure to yield a white residue. The residue was then subjected to silica gel column chromatography, using a gradient of methanol in dichloromethane (0-4%) which gave the desired product as a off-white solid (218 mg, 39.1%), R_{f} 0.5 (9:1 chloroform:methanol); HPLC (purity): 98.4%; mp 160-166° C.; ¹H-NMR (CDCl₃) δ (ppm): 8.31 (d, 1H), 8.22 (m, 2H), 7.88 (d, 1H), 7.63 (t, 1H), 7.35-7.23 (m, 3H), 6.81 (m, 2H), 4.15-4.10 (m, 2H), 4.05 (m, 1H), 3.74 (s, 3H), 2.94 (m, 2H), 2.31 (s, 3H), and 2.17 (m, 2H); IR (cm⁻¹) 3435.8, 2926.1, 2233.8, 1732.1, 1584.1, 1477.6, 1293.3, and 1267.1; MS m/z: 438.2[M+1]

The inhibition-activity data presented under sections TNF alpha, IL-6 and COX is only representative in nature.

### Tumor Necrosis Factor Alpha (TNF-α)

This assay determines the effect of the test compounds on the production of TNF-α in human whole blood. TNF-α assay is carried out as described by Armin Hatzelmann and Christian Schudt (J Pharm Exp Ther 297, 261, 2001). Compounds are tested for their ability to inhibit the activity of TNF-α in human whole blood. The test compounds are pre-incubated for 15 minutes at 37° C. and then stimulated with Lipopolysaccharide (Salmonella abortus equi, 1 □g/ml) for 4 hours at 37° C. in 5% CO₂. The levels of TNF-α are estimated using Enzyme linked Immunosorbent assay performed in a 96 well format as per the procedure of the manufacturer (Cayman Chemical, Ann Arbor, USA). Representative results of TNF-α inhibition are shown in the Table I.

TABLE I
% TNF-α Inhibitory Activity in (hPBMC)

| Examples | 10 μM | 1 μM |
|---|---|---|
| 2 | 54.32 | 6.87 |
| 3 | 26.95 | 3.42 |
| 4 | 26.15 | 19.78 |
| 5 | 52.42 | 18.43 |
| 10 | 50.51 | 19.85 |
| 11 | 47.49 | 11.67 |
| 15 | 41.17 | 48.14 |
| 22 | 47.99 | 53.07 |
| 26 | 2.67 | 20.77 |
| 27 | 9.77 | 3.51 |
| 31 | 28.53 | 10.79 |
| 36 | 18.81 | 8.81 |
| 37 | 42.11 | 14.54 |
| 39 | 45.80 | 26.06 |
| 44 | 45.61 | 23.48 |
| 45 | 32.08 | 12.27 |
| 47 | 26.53 | 18.04 |
| 48 | 22.65 | 20.70 |
| 56 | 54.34 | 26.51 |

### Interleukin-6 (IL-6)

This assay determines the effect of test compounds on the production of IL-6 from human whole blood. Compounds are tested for their ability to downregulate the production of IL-6 in activated whole blood. The test compounds are pre-incubated for 15 minutes at 37° C. and then stimulated with Lipopolysaccharide (Salmonella abortus equi, 1□g/ml) for 4 hours at 37° C. in 5% CO₂. The levels of IL-6 are estimated using Enzyme linked Immunosorbent assay performed in a 96 well format as per the procedure of the manufacturer. (Cayman Chemical, Ann Arbor, USA). Representative results of IL-6 inhibition are shown in the Table II.

TABLE II
% IL-6 Inhibitory Activity (hPBMC)

| Example | 10 μM | 1 μM |
|---|---|---|
| 2 | 52.74 | 17.19 |
| 3 | 17.71 | 13.13 |
| 25 | 12.51 | 22.40 |
| 37 | 32.23 | 21.12 |
| 38 | 25.99 | 19.09 |
| 65 | 30.63 | 13.71 |
| 39 | 19.09 | 28.54 |
| 40 | 21.30 | 20.46 |
| 41 | 3.09 | 7.63 |
| 42 | 18.48 | 7.96 |
| 44 | 22.73 | 13.17 |
| 46 | 17.93 | 29.93 |

### In-Vivo TNF-α Inhibition Assay

TNF-α inhibitory activity is assessed by in-vivo inhibition of serum TNF-α production in mice. This method is used to assess the inhibitory actions of compounds, on TNF-□ production in mouse (Griswold et al J Pharmacol Exp Ther 287,705, 1998, Garcia et al, Histol Histopathol 5(1), 43, 1990, and Victor et al, Physiol Res 52,789, 2003). Male Swiss albino mice with body weights equivalent within each group are selected. The animals are fasted for eighteen hours with free access to water. The control group receives only LPS and the drug treatment group receives LPS and the test compound. At the start of the experiment, the drug is administered orally. Thirty minutes later, the animals are given intraperitoneal injection with lipo-polysaccharide (LPS). Blood samples are withdrawn 90 minutes after the LPS challenge, which is the time point of maximal elevation of serum TNF-α activity. Blood was centrifuged for 10 minutes at 4° C. Serum samples were assayed for TNF-□ levels using Mouse ELISA kit. The Percent Inhibition of TNF-α production is determined by comparison with LPS-treated and LPS/drug treated groups.

TABLE III

| Example | % TNF-α inhibition in in vivo sepsis in mice |
|---|---|
| 17 | 82.69 |
| 62 | 86.10 |

### COX-1 and COX-2 Enzyme Based Assay

COX-1 and COX-2 enzyme based assays were carried out to check the inhibitory potential of test compounds on the production of prostaglandin by purified recombinant COX-1/COX-2 enzyme (Proc. Nat. Acad. Sci. USA, 88, 2692-2696, 1991; J. Clin. Immunoassay 15, 116-120, 1992) In this assay, the potential of the test compound to inhibit the production of prostaglandin's either by COX-1 or COX-2 from arachidonic acid (substrate) was measured. This was an enzyme based in-vitro assay to evaluate selective COX inhibition with good reproducibility.

Arachidonic acid was converted to PGH₂ (Intermediate product) by COX1/COX-2 in the presence or absence of the test compound. The reaction was carried out at 37° C. and after 2 minutes it was stopped by adding 1M HCl. The intermediate product PGH₂ was converted to a stable prostanoid product PGF_{2α} by SnCl₂ reduction. The amount of PGF_{2α} produced in the reaction was inversely proportional to the COX inhibitory potential of the test compound. The prostanoid product was quantified via enzyme immunoassay (EIA) using a broadly specific antibody that binds to all the major forms of prostaglandin, using Cayman ELISA kit as per the procedure outlined by the manufacturer (Cayman Chemicals, Ann Arbor, USA). Representative results of the COX enzyme inhibition are shown in the Table IV.

TABLE IV
Percentage Inhibition of COX enzymes in recombinanant enzyme assay

| | |
|---|---|
| COX-1 | COX-2 |
| (% inhibition) | (% inhibition) |

| Example | 1 μM | 10 μM | 1 μM | 10 μM |
|---|---|---|---|---|
| 17 | NA | NA | 41.48 | 42.74 |
| 20 | NA | 39.14 | 32.66 | 34.89 |
| 21 | NA | NA | 23.02 | 23.55 |
| 62 | NA | 16.45 | 41.97 | 36.45 |

We claim:

1. The present invention relates to novel compounds of the formula (I), 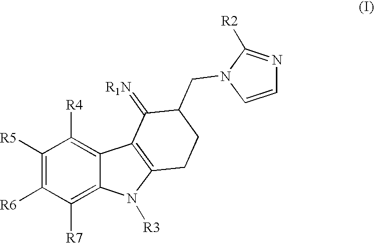
their derivatives, their analogs, their stereoisomers, their polymorphs, their hydrates, their solvates, their pharmaceutically acceptable salts, and compositions, wherein R¹ represents —O(CH₂)ₙR₈ where R₈ represents hydrogen; substituted or unsubstituted groups selected from (C₁-C₄) alkyl groups, such as methyl, ethyl, n-propyl, isopropyl, t-butyl and the like; alkenyl groups such as ethylene and the like, the alkenyl group may be substituted; alkynyl groups such as acetylene and the like, the alkynyl group may be substituted; aryl groups such as phenyl, naphthyl and the like, the aryl group may be substituted; aralkyl groups such as benzyl, phenylethyl, phenylpropyl and the like; the aralkyl group may be substituted; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like; the heteroaryl group may be substituted; haloalkyl groups selected from chloromethyl, chloroethyl, trifluoromethyl, trifluoroethyl, dichloromethyl, dichloroethyl and the like, the haloalkyl group may be substituted; cycloalkyl groups such as cyclopropyl, cyclobutyl, cyclopentyl and the like, the cycloalkyl group may be substituted; heterocyclyl containing at least one heteroatom selected from the O, N, S such as piperidine, piperazine, morpholine, 1,4-dioxane and the like, the heterocyclyl group may be substituted, or a counter ion, when R₈ represents —C(═O)R₉, therein R₉ represents hydrogen, hydroxyl, substituted or unsubstituted groups selected from (C₁-C₄) alkyl groups such as methyl, ethyl, n-propyl, isopropyl and the like; alkenyl groups such as ethylene and the like, the alkenyl group may be substituted; alkynyl groups such as acetylene and the like, the alkynyl group may be substituted; linear or branched (C₁-C₆) alkoxy groups, such as methoxy, ethoxy, n-propoxy, isopropoxy and the like, amino groups such as methyl amine, ethyl amine, isopropylamine, (N,N)-dimethyl amine and the like, aryl groups such as phenyl, naphthyl and the like, the aryl group may be substituted; arylalkoxy groups such as phenylmethoxy, phenylethoxy, phenylpropoxy, and the like; arylalkyl groups such as benzyl, phenylethyl, phenylpropyl and the like; aryl(C₂-C₆) alkenyl, aryl(C₂-C₆) alkynyl, (C₃-C₇) cycloalkyl, haloalkyl groups selected from chloromethyl, chloroethyl, trifluoromethyl, trifluoroethyl, dichloromethyl, dichloroethyl and the like; heteroaryl groups such as pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like, heteroarylalkoxy, heteroarylalkyl, heteroarylalkenyl wherein the alkenyl group is selected from ethylene and the like, and the hetero aryl part is selected from pyridyl, thienyl, furyl, pyrrolyl, oxazolyl, thiazolyl, imidazolyl, isooxazolyl, oxadiazolyl, triazolyl, thiadiazolyl, tetrazolyl, pyrazinyl, pyrimidinyl, quinolinyl, benzopyranyl, benzofuranyl, benzimidazolyl, benzoxazolyl, benzothiazolyl, benzopyrrolyl, benzoxadiazolyl, benzothiadiazolyl and the like, the heteroaryl group may be substituted; heteroaryl alkynyl, hetereoaryloxy, heterocyclyl, (heterocyclyl)alkenyl, (heterocyclyl)alkynyl wherein the heterocycle contains at least one hetroatom selected from the O, N, S such as piperidine, piperazine, pyrazine, morpholine, 1,4-dioxane and the like, (C₃-C₇) cycloalkyl groups such as cyclopropyl, cyclobutyl, cyclopentyl and the like.
R₂ represents hydrogen, hydroxyl, alkyl (selected from substituted or unsubstituted (C₁-C₄) alkyl groups, such as methyl, ethyl, n-propyl, isopropyl, t-butyl and the like), haloalkyl, halogen, mono or di alkylamino, nitro, alkoxy, thiol, alkylthio, aryl, aralkyl, arylthio, heteroaryl, heteroaralkyl, and cycloalkyl;
R₃ represents hydrogen, hydroxyl, nitro, nitroso, halogen, optionally substituted groups selected from alkyl (which may be selected from substituted or unsubstituted (C₁-C₄) alkyl groups, such as methyl, ethyl, n-propyl, isopropyl, t-butyl and the like), haloalkyl, mono or dialkylamino, alkoxy, arylalkyl, aryl, aryloxy heteroaryl, heteroaralkyl, cycloalkyl;
R₄, R₅, R₆ and R₇ may be same or different and independently represent hydrogen, nitro, hydroxy, formyl, azido, cyano, halo, or optionally substituted groups selected from alkyl, aryl, alkoxy, haloalkyl, hydrazine, monoalkylamino, dialkylamino, alkylsulfonyl, alkylsulfinyl, arylsulfonyl, arylsulfinyl, alkylthio, arylthio, arylalkyl, alkoxyalkyl, sulfamoyl, carboxylic acid and its derivatives;
When the aryl and heteroaryl groups representing R₈ and R₉ are substituted by one or more substituents which may be same or different, the substituents may be selected from halogens (fluorine, chlorine, bromine, iodine), hydroxy, nitro, cyano, azido, nitroso, amino, hydrazine, formyl, alkyl, haloalkyl, haloalkoxy, cycloalkyl, aryl (may be further substituted), alkoxy, aryloxy, acyl, acyloxy, acyloxyacyl, methylene dioxy, heterocyclyl, heteroaryl (may be further substituted), monoalkylamino, dialkylamino, acylamino, alkoxycarbonyl, aryloxycarbonyl, alkylsulfonyl, arylsulfonyl, alkylsulfinyl, arylsulfinyl, alkylthio, arylthio, sulfamoyl, alkoxyalkyl groups and carboxylic acids or its derivatives and these substituents are as defined above.
Furthermore, whenever the groups R₈ and R₉ represent substituted or unsubstituted 5 to 10 membered ring systems, the rings may be monocyclic or bicyclic, saturated or partially saturated or aromatic containing 1 to 4 heteroatoms selected from O, S and N.
n is an integer ranging from 0 to 2.

2. Novel compounds as claimed in the claim 1, derivatives, analogs, stereoisomers, polymorphs, hydrates, solvates, pharmaceutically acceptable salts, and compositions thereof.

3. Novel compounds as claimed in the claim 1, derivatives, analogs, stereoisomers, polymorphs, hydrates, solvates, pharmaceutically acceptable salts, and compositions are selected from:
1. (4E)-9-methyl-3-[(2-methyl-1H-imidazol-1-yl)methyl]-1,2,3,9-tetrahydro-4H-carbazol-4-one oxime;
2. (4E)-9-methyl-3-[(2-methyl-1H-imidazol-1-yl)methyl]-1,2,3,9-tetrahydro-4H-carbazol-4-one O-benzyloxime;
3. (4E)-9-methyl-3-[(2-methyl-1H-imidazol-1-yl)methyl]-1,2,3,9-tetrahydro-4H-carbazol-4-one O-ethyloxime;
4. (4E)-9-methyl-3-[(2-methyl-1H-imidazol-1-yl)methyl]-1,2,3,9-tetrahydro-4H-carbazol-4-one O-2,2,2-trifluoroethyloxime;
5. (4E)-9-methyl-3-[(2-methyl-1H-imidazol-1-yl)methyl]-1,2,3,9-tetrahydro-4H-carbazol-4-one O-(4-methoxybenzyl)oxime;
6. (4E)-9-methyl-3-[(2-methyl-1H-imidazol-1-yl)methyl]-1,2,3,9-tetrahydro-4H-carbazol-4-one O-(allyl)oxime;
7. (4E)-9-methyl-3-[(2-methyl-1H-imidazol-1-yl)methyl]-1,2,3,9-tetrahydro-4H-carbazol-4-one O-(prop-2-ynyl)oxime;
8. (4E)-9-methyl-3-[(2-methyl-1H-imidazol-1-yl)methyl]-1,2,3,9-tetrahydro-4H-carbazol-4-one O-(3-cyano-pyridin-2-yl)oxime;
9. (4E)-9-methyl-3-[(2-methyl-1H-imidazol-1-yl)methyl]-1,2,3,9-tetrahydro-4H-carbazol-4-one O-(4-fluorobenzyl)oxime;
10. (4E)-9-methyl-3-[(2-methyl-1H-imidazol-1-yl)methyl]-1,2,3,9-tetrahydro-4H-carbazol-4-one O-(pyidin-3-ylmethyl)oxime;
11. (4E)-9-methyl-3-[(2-methyl-1H-imidazol-1-yl)methyl]-1,2,3,9-tetrahydro-4H-carbazol-4-one O-(pyridin-4-ylmethyl)oxime;
12. (4E)-9-methyl-3-[(2-methyl-1H-imidazol-1-yl)methyl]-1,2,3,9-tetrahydro-4H-carbazol-4-one O-(3,5-dimethyl-4-methoxy-pyridin-2-ylmethyl)oxime;
13. (4E)-9-methyl-3-[(2-methyl-1H-imidazol-1-yl)methyl]-1,2,3,9-tetrahydro-4H-carbazol-4-one O-(pyridin-2-ylmethyl)oxime;
14. (4E)-9-methyl-3-[(2-methyl-1H-imidazol-1-yl)methyl]-1,2,3,9-tetrahydro-4H-carbazol-4-one O-(4-cyanobenzyl)oxime;
15. (4E)-9-methyl-3-[(2-methyl-1H-imidazol-1-yl)methyl]-1,2,3,9-tetrahydro-4H-carbazol-4-one O-(4-nitrobenzyl)oxime;
16. (4E)-9-methyl-3-[(2-methyl-1H-imidazol-1-yl)methyl]-1,2,3,9-tetrahydro-4H-carbazol-4-one O-(5-nitropyridin-2-yl)oxime;
17. (4E)-9-methyl-3-[(2-methyl-1H-imidazol-1-yl)methyl]-1,2,3,9-tetrahydro-4H-carbazol-4-one O-[5-bromo-pyridin-2-yl]oxime;
18. (4E)-9-methyl-3-[(2-methyl-1H-imidazol-1-yl)methyl]-1,2,3,9-tetrahydro-4H-carbazol-4-one O-[3-cyano-5,6-dimethylpyridin-2-yl]oxime;
19. (4E)-9-methyl-3-[(2-methyl-1H-imidazol-1-yl)methyl]-1,2,3,9-tetrahydro-4H-carbazol-4-one O-[3-nitro-pyridin-2yl]oxime;
20. (4E)-9-methyl-3-[(2-methyl-1H-imidazol-1-yl)methyl]-1,2,3,9-tetrahydro-4H-carbazol-4-one O-(2,6-dimethoxypyrimidin-4-yl)oxime;
21. (4E)-9-methyl-3-[(2-methyl-1H-imidazol-1-yl)methyl]-1,2,3,9-tetrahydro-4H-carbazol-4-one O-[5-trifluoromethyl-pyridin-2-yl]oxime;
22. (4E)-9-methyl-3-[(2-methyl-1H-imidazol-1-yl)methyl]-1,2,3,9-tetrahydro-4H-carbazol-4-one O-(-chloro-5-trifluoromethyl-pyridin-2-yl)oxime.
23. (4E)-9-methyl-3-[(2-methyl-1H-imidazol-1-yl)methyl]-1,2,3,9-tetrahydro-4H-carbazol-4-one O-(3-cyano-6-methylpyridin-2-yl)oxime;
24. (4E)-9-methyl-3-[(2-methyl-1H-imidazol-1-yl)methyl]-1,2,3,9-tetrahydro-4H-carbazol-4-one O-(6-trifluoromethylpyrimidin-2-yl)oxime;
25. (4E)-9-methyl-3-[(2-methyl-1H-imidazol-1-yl)methyl]-1,2,3,9-tetrahydro-4H-carbazol-4-one O-acetyloxime;
26. (4E)-9-methyl-3-[(2-methyl-1H-imidazol-1-yl)methyl]-1,2,3,9-tetrahydro-4H-carbazol-4-one O-trifluoroacetyloxime;
27. (4E)-9-methyl-3-[(2-methyl-1H-imidazol-1-yl)methyl]-1,2,3,9-tetrahydro-4H-carbazol-4-one O-2-chloropyridinoyloxime;
28. (4E)-9-methyl-3-[(2-methyl-1H-imidazol-1-yl)methyl]-1,2,3,9-tetrahydro-4H-carbazol-4-one O-cyclobutanoyloxime;
29. (4E)-9-methyl-3-[(2-methyl-1H-imidazol-1-yl)methyl]-1,2,3,9-tetrahydro-4H-carbazol-4-one O-2-cyclopentanoyloxime;
30. (4E)-9-methyl-3-[(2-methyl-1H-imidazol-1-yl)methyl]-1,2,3,9-tetrahydro-4H-carbazol-4-one O-benzoyloxime;
31. (4E)-9-methyl-3-[(2-methyl-1H-imidazol-1-yl)methyl]-1,2,3,9-tetrahydro-4H-carbazol-4-one O-4-chloro-benzoyloxime;
32. (4E)-9-methyl-3-[(2-methyl-1H-imidazol-1-yl)methyl]-1,2,3,9-tetrahydro-4H-carbazol-4-one O-cyclopropanoyloxime;
33. (4E)-9-methyl-3-[(2-methyl-1H-imidazol-1-yl)methyl]-1,2,3,9-tetrahydro-4H-carbazol-4-one O-2-fluorobenzoyloxime;
34. (4E)-9-methyl-3-[(2-methyl-1H-imidazol-1-yl)methyl]-1,2,3,9-tetrahydro-4H-carbazol-4-one O-4-trifluoromethylphenyloxime;
35. (4E)-9-methyl-3-[(2-methyl-1H-imidazol-1-yl)methyl]-1,2,3,9-tetrahydro-4H-carbazol-4-one O-4-methoxybenzoyloxime;
36. (4E)-9-methyl-3-[(2-methyl-1H-imidazol-1-yl)methyl]-1,2,3,9-tetrahydro-4H-carbazol-4-one O-4-methylbenzoyloxime;
37. (4E)-9-methyl-3-[(2-methyl-1H-imidazol-1-yl)methyl]-1,2,3,9-tetrahydro-4H-carbazol-4-one O-2-chlorobenzoyloxime;
38. (4E)-9-methyl-3-[(2-methyl-1H-imidazol-1-yl)methyl]-1,2,3,9-tetrahydro-4H-carbazol-4-one O-4-fluorobenzoyloxime;
39. (4E)-9-methyl-3-[(2-methyl-1H-imidazol-1-yl)methyl]-1,2,3,9-tetrahydro-4H-carbazol-4-one O-2-bromobenzoyloxime;
40. (4E)-9-methyl-3-[(2-methyl-1H-imidazol-1-yl)methyl]-1,2,3,9-tetrahydro-4H-carbazol-4-one O-6-chloropyridinoyloxime;
41. (4E)-9-methyl-3-[(2-methyl-1H-imidazol-1-yl)methyl]-1,2,3,9-tetrahydro-4H-carbazol-4-one O-4-ethylbenzoyloxime;
42. (4E)-9-methyl-3-[(2-methyl-1H-imidazol-1-yl)methyl]-1,2,3,9-tetrahydro-4H-carbazol-4-one O-cyclohexanoyloxime;
43. (4E)-9-methyl-3-[(2-methyl-1H-imidazol-1-yl)methyl]-1,2,3,9-tetrahydro-4H-carbazol-4-one O-(3-thiophenyl)propenoyloxime;
44. (4E)-9-methyl-3-[(2-methyl-1H-imidazol-1-yl)methyl]-1,2,3,9-tetrahydro-4H-carbazol-4-one O-4-cyanobenzoyloxime;
45. (4E)-9-methyl-3-[(2-methyl-1H-imidazol-1-yl)methyl]-1,2,3,9-tetrahydro-4H-carbazol-4-one O-5-bromo-2-furanoyloxime;
46. (4E)-9-methyl-3-[(2-methyl-1H-imidazol-1-yl)methyl]-1,2,3,9-tetrahydro-4H-carbazol-4-one O-3-quinolinoyloxime;
47. (4E)-9-methyl-3-[(2-methyl-1H-imidazol-1-yl)methyl]-1,2,3,9-tetrahydro-4H-carbazol-4-one O-5-nitro-2-furanoyloxime;
48. (4E)-9-methyl-3-[(2-methyl-1H-imidazol-1-yl)methyl]-1,2,3,9-tetrahydro-4H-carbazol-4-one O-3-methyl-2-furanoyloxime;
49. (4E)-9-methyl-3-[(2-methyl-1H-imidazol-1-yl)methyl]-1,2,3,9-tetrahydro-4H-carbazol-4-one O-3-methyl-2-thiophenoyloxime;
50. (4E)-9-methyl-3-[(2-methyl-1H-imidazol-1-yl)methyl]-1,2,3,9-tetrahydro-4H-carbazol-4-one O-5-methyl-3-isoxazoloyloxime;
51. (4E)-9-methyl-3-[(2-methyl-1H-imidazol-1-yl)methyl]-1,2,3,9-tetrahydro-4H-carbazol-4-one O-5-methyl-2-thiophenoyloxime;
52. (4E)-9-methyl-3-[(2-methyl-1H-imidazol-1-yl)methyl]-1,2,3,9-tetrahydro-4H-carbazol-4-one O-3-methyl-5-isoxazoloyloxime;
53. (4E)-9-methyl-3-[(2-methyl-1H-imidazol-1-yl)methyl]-1,2,3,9-tetrahydro-4H-carbazol-4-one O-3-thiophenoyloxime;
54. (4E)-9-methyl-3-[(2-methyl-1H-imidazol-1-yl)methyl]-1,2,3,9-tetrahydro-4H-carbazol-4-one O-4-methylphenylacetyloxime;
55. (4E)-9-methyl-3-[(2-methyl-1H-imidazol-1-yl)methyl]-1,2,3,9-tetrahydro-4H-carbazol-4-one O-2-thiophenoyloxime;
56. (4E)-9-methyl-3-[(2-methyl-1H-imidazol-1-yl)methyl]-1,2,3,9-tetrahydro-4H-carbazol-4-one O-5-chloro-2-thiophenoyloxime;
57. (4E)-9-methyl-3-[(2-methyl-1H-imidazol-1-yl)methyl]-1,2,3,9-tetrahydro-4H-carbazol-4-one O-[(4,5-dibromo-thien-2-yl)carbonyl]oxime;
58. (4E)-9-methyl-3-[(2-methyl-1H-imidazol-1-yl)methyl]-1,2,3,9-tetrahydro-4H-carbazol-4-one O-[(6-methylpyrazin-2-yl)carbonyl]oxime;
59. (4E)-9-methyl-3-[(2-methyl-1H-imidazol-1-yl)methyl]-1,2,3,9-tetrahydro-4H-carbazol-4-one O-[(pyridin-2-yl)carbonyl]oxime;
60. (4E)-9-methyl-3-[(2-methyl-1H-imidazol-1-yl)methyl]-1,2,3,9-tetrahydro-4H-carbazol-4-one [(furan-2-yl)carbonyl]oxime;
61. (4E)-9-methyl-3-[(2-methyl-1H-imidazol-1-yl)methyl]-1,2,3,9-tetrahydro-4H-carbazol-4-one O-[(2E)-3-(1,3-benzodioxol-5-yl)prop-2-enoyl]oxime;
62. (4E)-9-methyl-3-[(2-methyl-1H-imidazol-1-yl)methyl]-1,2,3,9-tetrahydro-4H-carbazol-4-one O-[(5-bromothien-2-yl)carbonyl]oxime;
63. (4E)-9-methyl-3-[(2-methyl-1H-imidazol-1-yl)methyl]-1,2,3,9-tetrahydro-4H-carbazol-4-one [(furan-3-yl)carbonyl]oxime;
64. 4-(hydroxyamino)-9-methyl-3-[(2-methyl-1H-imidazol-1-yl)methyl]-2,3,4,9-tetrahydro-1H-carbazole; and
65. (4E)-9-methyl-3-[(2-methyl-1H-imidazol-1-yl)methyl]-1,2,3,9-tetrahydro-4H-carbazol-4-one O-3-cyanobenzoyloxime;

4. A pharmaceutical composition, which comprises a compound of formula (I) 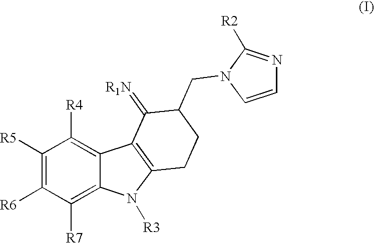
as defined in the claim 1 and a pharmaceutically acceptable carrier, diluent, excipient or solvate.

5. A pharmaceutical composition as claimed in the claim 1, in the form of a tablet, capsule, powder, syrup, solution or suspension.

6. A pharmaceutical composition as claimed in claim 4, wherein the amount of the compound of claim 1 in the composition is less than 60% by weight.

7. A pharmaceutical composition comprising a therapeutically effective amount of a compound of claims 1 to 3 in the form of a tablet, capsule, powder, syrup, solution or suspension to treat 5-HT3 antagonists and as anti-emetic agents.

8. A method of prophylaxis or treatment of asthma; COPD; psychotic diseases; nausea and vomiting; treatment of alcohol dependency; rheumatoid arthritis; osteoporosis; multiple myeloma; uveititis; acute and chronic myelogenous leukemia; ischemic heart disease; atherosclerosis; cancer; ischemic-induced cell damage; pancreatic beta cell destruction; osteoarthritis; rheumatoid spondylitis; gouty arthritis; inflammatory bowel disease; adult respiratory distress syndrome (ARDS); psoriasis; Crohn's disease; allergic rhinitis; ulcerative colitis; anaphylaxis; contact dermatitis; muscle degeneration; cachexia; type I and type II diabetes; bone resorption diseases; ischemia reperfusion injury; brain trauma; multiple sclerosis; cerebral malaria; sepsis; septic shock; toxic shock syndrome; fever, and myalgias due to infection, HIV-1, HIV-2, HIV-3, cytomegalovirus (CMV), influenza, adenovirus, the herpes viruses (including HSV-1, HSV-2), and herpes zoster infection in a mammal comprising administering an effective amount of, a compound of claims 1 to 3, to the mammal in need thereof.

9. A method of lowering plasma concentrations of either or both TNF-α and IL-1 comprising administering an effective amount of a compound of claims 1 to 3, to the mammal in need thereof.

10. A method of lowering plasma concentrations of either or both IL-6 and IL-8 comprising administering an effective amount of, a compound to any one of claims 1 to 3, to the mammal in need thereof.

11. A method of lowering plasma concentrations of anyone or a combination or all of TNF-α and IL (1, 1β, 2, 4, 5, 6, 8, 10, 12, 13, 15, 18, 23) comprising administering an effective amount of a compound to any one of claims 1 to 3, to the mammal in need thereof.

12. A method of prophylaxis or treatment of a pain disorder in a mammal comprising administering an effective amount of, a compound to any one of claims 1 to 3, to the mammal in need thereof.

13. A method of decreasing prostaglandin production in a mammal comprising administering an effective amount of, a compound to any one of claims 1 to 3, to the mammal in need thereof.
